(19) United States
(12) Patent Application Publication
(10) Pub. No.: US 2012/0114675 A1
(43) Pub. Date: May 10, 2012

(54) FOXP3+ NATURAL KILLER T-CELLS AND THE TREATMENT OF IMMUNE RELATED DISEASES

(76) Inventors: Luis Ricardo Simoes da Silva Graca, Lisboa (PT); Maria Sofia Ferreira Monteiro, Lisboa (PT)

(21) Appl. No.: 13/129,146

(22) PCT Filed: Nov. 13, 2009
(86) PCT No.: PCT/PT2009/000060

§ 371 Date: May 13, 2011

Related U.S. Application Data
(60) Provisional application No. 61/114,362, filed on Nov. 13, 2008.

(30) Foreign Application Priority Data
Sep. 25, 2009 (PT) 104764

(51) Int. Cl. A61K 35/14; A61K 35/00; A61P 29/00; A61P 11/06; A61P 37/06; C12N 5/0783; A61P 1/00

(52) U.S. Cl. 424/184.1; 435/375; 424/278.1

(57) ABSTRACT

In one aspect, the invention provides isolated populations of cells comprising Foxp3+ natural killer T-cells, methods of generating Foxp3+ natural killer T-cells and methods for suppressing the immune response in specific organs, including the liver and the lungs.

### RELATED APPLICATIONS

This application claims the benefit under 35 U.S.C. §119(e) from U.S. provisional applications Ser. No. 61/114,362, entitled “Foxp3+ Regulatory natural killer T cells and a method for their generation” filed Nov. 13, 2008.

This application claims the benefit of Portuguese Provisional Application number 104764, entitled “New process and use of cells” filed Sep. 25, 2009.

The entire contents of each of the above indicated applications are herein incorporated by reference.

### BACKGROUND OF THE INVENTION

In recent years, the role of NKT cells in modulating the immune system has gained significant attention. NKT-cell function relies on cell killing and, most of all, on cytokine release. In particular, NKT cells have been shown capable of producing cytokines characteristic of Th1, Th2, or Th17 responses, therefore influencing adaptive immunity (1)(2)(3)(4)(5)(6)(7). The impact on conventional T cells translates into a diversity of immune pathologies that are suppressed or exacerbated by NKT cells (8)(9)(10)(11)(12)(13). This NKT cell influence on adaptive immunity has been reported in transplantation (14), allergy (15), autoimmunity (16)(17), and other inflammatory pathologies (18)(19).

### SUMMARY OF THE INVENTION

In one aspect, the invention provides an isolated Foxp3+ natural killer T cell, and populations of cells comprising Foxp3+ natural killer T cells. In one aspect, the invention also provides methods for generating Foxp3+ natural killer T cells using TGF-β and one or more NKT stimulants. The Foxp3+ natural killer T cells can be generated in vitro and they can be generated in situ in a subject. It is shown herein that Foxp3+ natural killer T cells have similar immunosuppressive properties to Treg cells and the Foxp3+ natural killer T cells can therefore be used to treat a variety of immune disorders and conditions.

Foxp3+ natural killer T cells, when administered systemically, home to the liver and the lungs. In one aspect, the invention therefore provides methods for the treatment of immune disorders and conditions in the liver and the lung by systemic administration of Foxp3+ natural killer T cells. These immune disorders and conditions include graft versus host disease, unwanted side effects associated with or caused by liver transplant and islet transplantation, and asthma. The homing of Foxp3+ natural killer T cells to the liver and the lungs also allows for the administration of therapeutic polypeptides and other agents to these organs.

It is further shown herein that Foxp3+ natural killer T cells can be generated in situ in a specific anatomical location (e.g. an organ) through local administration of TGF-β and NKT stimulants. It is also shown herein that either TGF-β or NKT stimulants do not need to be administered if available in sufficient amount in the specific anatomical location. Thus, in one aspect the invention provides methods for the treatment of immune disorders and conditions in a specific anatomical location through the local in situ generation of Foxp3+ natural killer T cells

In one aspect the invention provides an isolated Foxp3+ natural killer T-cell.

In one aspect the invention provides an isolated population of cells comprising: (a) at least 0.001% Foxp3+ natural killer T-cells, or (b) at least 10 Foxp3+ natural killer T-cells. In some embodiments, the percentage of Foxp3+ natural killer T-cells is at least 0.001%, at least 0.01%, at least 0.05%, at least 0.1%, at least 0.5%, at least 1%, at least 5%, at least 10%, at least 20%, at least 30%, at least 40%, at least 50%, at least 60%, at least 70%, at least 80%, or at least 90%. In some embodiments, the number of Foxp3+ natural killer T-cells is at least 1, at least 10, at least 50, at least 100, at least 500, at least 1,000, at least 5,000, at least 10,000, at least 50,000, at least 100,000, at least 1×10⁶, at least 1×10⁷, or at least 1×10⁸ cells. In some embodiments, the population of cells is a population of blood cells, a population of leukocytes, a population of T-cells, or a population of natural killer T-cells. In some embodiments, the population of cells is a population of T-cells.

In one aspect the invention provides a method of generating a Foxp3+ natural killer T-cell, the method comprising contacting a population of cells comprising natural killer T-cells with a combination of TGF-β and one or more NKT-stimulants in amounts sufficient to generate a Foxp3+ natural killer T-cell. In some embodiments, the method further comprises contacting the population of cells with IL-2. In some embodiments, the method further comprises contacting the population of cells with any one or any combination of IL-7, IL-15 and IL-2. In some embodiments, the method further comprises contacting the population of cells with any one or any combination of neutralizing antibodies selected from the group consisting of anti-IFNγ, anti-IL-4, anti-IL-6, anti-IL12 and anti-IL-27. In some embodiments, the population of cells is a population of blood cells, a population of leukocytes, a population of T-cells, or a population of natural killer T-cells. In some embodiments, the population of cells is harvested from a subject.

In one aspect the invention provides a method of increasing the number of Foxp3+ natural killer T-cells, the method comprising contacting a population of cells comprising at least one Foxp3+ natural killer T-cell with a combination of TGF-β, an NKT-stimulant, one or more proliferation inducing cytokines, and one or more neutralizing antibodies in amounts sufficient to increase the number of Foxp3+ natural killer T-cells. In some embodiments, the number of Foxp3+ natural killer T-cells is increased by at least 2-fold, by at least 5-fold, by at least 10-fold, by at least 50-fold, by at least 100-fold, by at least 200-fold, by at least 500-fold, by at least 1000-fold, by at least 10,000-fold, by at least 100,000-fold, by at least 10⁶-fold, by at least 10⁷-fold. In some embodiments, the proliferation inducing cytokine is one or any combination of IL-2, IL-7, IL-15 and IL-21. In some embodiments, the neutralizing antibody is any one or any combination of anti-IFNγ, anti-IL-4, anti-IL-6, anti-IL 12 and anti-IL-27.

In one aspect the invention provides a method for delivering a natural killer T-cell to the liver or to mucosal tissue in a subject, the method comprising: administering systemically Foxp3+ natural killer T-cells to the subject. In one aspect the invention provides a method for delivering a natural killer T-cell to the liver or to mucosal tissue in a subject, the method comprising administering locally Foxp3+ natural killer T-cells to the subject. In some embodiments, the Foxp3+ natural killer T-cells are autologous cells. In some embodiments, the Foxp3+ natural killer T-cells are generated by contacting natural killer T-cells with one or more NKT-cell stimulants and TGF-β in amounts sufficient to generate Foxp3+ natural killer T-cells. In some embodiments, the Foxp3+ natural killer T-cells are administered in an amount effective to suppress an immune response in the liver or mucosal tissue. In some embodiments, the suppressing of the immune response in the liver is to treat graft versus host disease, unwanted immune responses caused by or associated with islet transplantation, unwanted immune responses caused by or associated with liver transplant or immune-mediated inflammation to the liver. In some embodiments, the Foxp3+ natural killer T-cells are administered in conjunction with islet transplantation or liver transplant. In some embodiments, the genome of the Foxp3+ natural killer T-cell comprises a nucleic acid encoding a polypeptide, and wherein the delivery of the Foxp3+ natural killer T-cell to the liver results in the expression of the polypeptide in the liver.

In one aspect the invention provides a method for suppressing an immune response in an organ in a subject, the method comprising delivering locally to the organ one or more NKT-stimulants in an amount sufficient to suppress the immune response in the organ. In some embodiments, the method further comprises delivering locally to the organ TGF-β in an amount sufficient to suppress the immune response in the organ. In some embodiments, the immune response is an immune response to an antigen. In some embodiments, the immune response is an autoimmune response. In some embodiments, the organ is the gut, or the lungs. In some embodiments, the suppression of the immune response is to treat inflammatory bowel disease, Crohn's disease, or asthma. In some embodiments, the delivering locally to the organ is delivery to mucosal tissue.

In one aspect the invention provides a pharmaceutical composition comprising a population of cells comprising Foxp3+ natural killer T-cells. In some embodiments, the population of cells is a population of blood cells. In some embodiments, the population of cells is a population of leukocytes. In some embodiments, the population of cells is a population of T-cells. In some embodiments, the population of cells is a population of NKT cells.

In one aspect the invention provides a pharmaceutical composition comprising TGF-β and one or more NKT stimulants.

Each of the limitations of the invention can encompass all of the various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including”, “comprising”, “having”, “containing”, “involving”, and variations thereof herein, is meant to encompass the items listed thereafter and equivalents thereof as well as additional items.

### BRIEF DESCRIPTION OF THE DRAWINGS

The figures are illustrative only and are not required for enablement of the invention disclosed herein.

FIG. 1 shows that iNKT cells upregulate Foxp3 expression in presence of TGF-β. (a) iNKT and CD4⁺CD25⁻ T cells from the spleen of C57Bl/6 or Foxp3^{gfp} knock-in mice were FACS sorted and cultured for 3 days in the presence of IL-2 and IL-15 with or without addition of TGF-β. The left dotplots show the gating strategy used for cell sorting of iNKT cells, as well as the background control staining assessed with an empty CD1d tetramer. The gating strategy used for analysis, after culture, is also depicted for iNKT cells (upper panel) and CD4 T cells gated in the TCRβ⁺ population (lower panel). Foxp3 expression was assessed by intracellular staining in iNKT (upper row) and control CD4 T-cell cultures (lower row) isolated from C57Bl/6 mice, or through GFP fluorescence in cells isolated from Foxp3^{gfp} knock-in mice. Dotplots shown are representative of triplicate cultures from three independent experiments. (b) Thymic iNKT cells from C57Bl/6 (top) and Foxp3^{gfp} knock-in mice (bottom) were sorted, cultured in the presence of IL-2, IL-15 and TGF-β, and analyzed for Foxp3 expression as described. (c) Foxp3 expression by iNKT cells was confirmed at single-cell level by confocal microscopy. Foxp3^{gfp} cells were FACS sorted after 3 days of culture, their invariant TCR was re-stained with PE-labeled CD1d/PBS57 tetramer (red) and the nucleus counterstained with DAPI (blue). Foxp3 expression fluoresces in green. (d) iNKT and CD4⁺CD25⁻ T cells of C57Bl/6 mice were isolated from the spleen and cultured for 3 days in presence of 5 ng/mL of IL-2 and different concentrations of TGF-β. Cultures were set in duplicate and results are representative of three independent experiments. (e) Splenic iNKT and CD4⁺CD25⁻ T cells (not shown) were cultured for 3 days with different concentrations of plate-bound anti-CD3. Dotplots shown are representative of triplicate cultures from three independent experiments.

FIG. 2 shows cultures of Balb/c iNKT cells under different cytokine conditions. iNKT cells were isolated from the spleen of Balb/c mice, FACS sorted according to the co-expression of a CD1d/PBS57 tetramer and a pan TCR-β MAb, and stimulated with 3 μg/mL of plate-bound anti-CD3 for 3 days in the indicated conditions. Results are representative of three independent experiments.

FIG. 3 shows the phenotype and in vivo stability of Foxp3+ iNKT cells. Murine iNKT and CD4⁺CD25⁻ cells were FACS sorted, cultured for 3 days in the presence of TGF-β and IL-2 and co-stained for Foxp3 and the indicated molecules. (a and b) The profiles depicted were gated inside the iNKT or CD4-cell region (defined as illustrated in FIG. 6). Results are representative of triplicates from at least two independent experiments. (c) RT-PCR of the mRNAs coding for Foxp3 and PLZF from FACS sorted iNKT or CD4 T cells after 3 days of culture in the presence of TGF-β and IL-2. The expression of each gene is presented relative to EF1A expression.

FIG. 4 shows the in vivo stability of Foxp3+ iNKT cells. Splenic iNKT and CD4⁺CD25⁻ T cells were isolated from Foxp3^{gfp} knockin mice and cultured for 3 days in presence of TGF-β and IL-2. Foxp3-GFP⁺ cells were sorted by FACS and 5×10⁴ iNKT (upper panels) or CD4⁺CD25⁻ T cells (bottom panels) were injected i.v. into RAG2^{-/-} recipient hosts. These mice were sacrificed after 21 days and the presence of iNKT and CD4 T cells was assessed in several organs. The figure shows Foxp3 and CD25 expression of iNKT and CD4 T cells in the liver and in a pool of lymph nodes (pLNs). iNKT cells could be only detected in the liver.

FIG. 5 shows that Foxp3+ iNKT cells suppress T-cell proliferation through a GITR-mediated contact-dependent mechanism. (a) Foxp3+ iNKT, Foxp3⁻ iNKT or iTreg cells were sorted from in vitro cultures of iNKT and CD4 T cells from Foxp3^{gfp} knock-in mice under polarizing conditions, and nTreg cells were isolated from naïve C57Bl/6 mice. The sorted cells were co-cultured in triplicate, at different ratios, with mitomycin C-treated splenocytes and FACS-sorted CD4⁺CD25⁻ (“responder”) T cells for 96 hours in the presence of soluble anti-CD3 MAb. Proliferation was assessed through [³H]thymidine incorporation in the last 12 h of culture. Top panel depicts the average of proliferation inhibition from three independent experiments (each one with triplicates) normalized to the proliferation of responder cells alone (mean±SEM). Bottom panel shows data from a representative experiment at a ratio of 2:1 (8000 regulatory cells to 4000 responders). The inhibition of proliferation was statistically significant (n=3, *P<0.05). (b) Addition of anti-GITR, but not anti-IL10R blocking antibody abrogated the suppressive effect (n=4, **P<0.01). (c) Foxp3+ iNKT cells were cultured in transwells at 1:1 ratio with responder cells stimulated as in (a). The black histogram represents responder cell proliferation when these were cultured with Foxp3+ iNKT cells in the same well; gray histogram represents responder cell proliferation when these were cultured in a separate well from Foxp3+ iNKT cells; dotted histogram represents responder cell proliferation in the absence of regulatory cells. Percentages indicate the frequency of responder cells from the three conditions within the indicated gate.

FIG. 6 shows that in vivo conversion of Foxp3+ iNKT cells is TGF-β dependent. (a) Analysis of Foxp3 expression in lymphocytes isolated from the liver, pooled lymph nodes, Peyer's Patches, spleen and thymus of naïve C57Bl/6 mice. iNKT cells were identified by co-staining with a CD tetramer loaded with PBS57 and a pan TCR-β antibody inside the lymphocyte gate. Background staining of iNKT cells was assessed for every organ with parallel stainings with an empty CD1d tetramer (shown for liver only). (b) Foxp3 expression was analyzed through intracellular antibody staining. Foxp3+ cells were only present among the CD1d/PBS57⁻ cells. We confirmed these Foxp3+ cells as being CD4⁺ lymphocytes (right dotplot, shown only for LNs). Results are representative of three mice from two independent experiments. (c) Analysis of Foxp3 expression in iNKT cells from the MLNs of C57BL/6 or dnTGFβRII mice exposed to intra-gastric delivery of α-GalCer over one week (gated on iNKT cells). (d) Analysis of Foxp3 expression, as well as the house keeping gene EF1A in iNKT or CD4 T cells isolated from the lungs of mice with chronic or acute allergic airways disease. From the lungs of each animal between 1,000 and 5,000 iNKT cells were sorted by flow cytometry, and are represented in individual lanes. Lung iNKT and CD4 T cells from five unmanipulated naïve control mice were pooled together, due to lower cell numbers. The experiment was performed independently in C57Bl/6 and BALB/c mice.

FIG. 7 shows that iNKT cells from naive Balb/c and Foxp3^{gfp} knock-in mice lack Foxp3 expression. Analysis of Foxp3 expression in lymphocytes isolated from the liver, a pool of lymph nodes, Peyer's Patches, spleen and thymus of Balb/c mice and Foxp3^{gfp} naïve mice. iNKT cells were identified by co-staining with a CD1d tetramer loaded with PBS57 and a pan TCR-β antibody inside the lymphocyte gate.

FIG. 8 shows the isolation of iNKT cells from the lungs of Balb/c and C57Bl/6 mice with allergic airways disease. Groups of 5 female Balb/c or C57Bl/6 mice were sensitized with OVA-alum i.p. and received an intranasal challenge with 50 μg OVA in saline on the indicated days in order to induce a chronic or acute form of allergic airways disease. (a) Schematic representation of the protocol followed to induce chronic and acute allergic airways disease. (b) Eosinophilia in the bronchoalveolar lavage (BAL) and cellular content of the lungs. (c) FACS data depicting the iNKT and CD4 T-cell staining in the lungs of one representative mouse from each experimental group, showing the gates used to sort iNKT (left column) and CD4 T cells (right), after doublet exclusion (not shown), prior to quantitative RT-PCR analysis. (d) Histological sections of lung tissue stained with hematoxylin/eosin from chronic (upper panel), acute (middle panel) and naïve mice (lower panel).

FIG. 9 shows that Foxp3+ iNKT cells accumulate in the cervical lymph nodes of mice protected from EAE. EAE was induced in C57Bl/6 mice by co-administration of MOG peptide with CFA and pertussis toxin. Some mice were simultaneously treated with α-GalCer as described elsewhere²⁶. EAE was clinically scored and central nervous system infiltrates, as well as lymphocyte populations of the cervical lymph nodes (LNs) and spleen were evaluated. (a) Left panels show the iNKT population in the cervical LNs and right panels show Foxp3 expression inside the iNKT gate. (b) The absolute number of iNKT cells (top panel) and Foxp3+ iNKT cells (bottom panel) is depicted, each symbol corresponding to one individual mouse from the naïve, MOG-induced and MOG+α-GalCer treated experimental groups. Horizontal thin bars represent the average and horizontal thick bars indicate the statistical significance between groups (n=4 or 5, *P<0.05).

FIG. 10 shows that Foxp3 expression can be induced in human iNKT cells. Human iNKT and CD4⁺ T cells from peripheral blood were magnetically enriched and co-cultured for 5 days in the presence or absence of a conversion cocktail including TGF-β, anti-IL4, anti-IL12, anti-IFN-γ and anti-CD28 MAbs. iNKT cells were identified by co-staining of the human CD1d tetramer loaded with PBS57 and an anti-TCR-Vβ11 MAb inside the lymphocyte gate (top). Background staining of iNKT cells was evaluated in parallel stainings with an empty human CD1d tetramer (top left). CD4 T cells were gated inside the CD1d/PBS57-negative region (top right). Lower panels show the co-expression of Foxp3 along with CD25, CD127, GITR or CD 161 in iNKT (upper row) and CD4⁺ T-cell gate (lower row). Results are representative of three independent experiments from different blood donors with at least three replicate cultures per condition.

### DETAILED DESCRIPTION OF THE INVENTION

In one aspect, the invention provides an isolated Foxp3+ natural killer T cell, and populations of cells comprising Foxp3+ natural killer T cells. In one aspect, the invention provides methods for generating Foxp3+ natural killer T cells using TGF-β and one or more NKT stimulants. The Foxp3+ natural killer T cells can be generated in vitro and they can be generated in situ in a subject. It is shown herein that Foxp3+ natural killer T cells have similar immunosuppressive properties to Treg cells and that the Foxp3+ natural killer T cells can therefore be used to treat a variety of immune disorders and conditions.

### Foxp3+ Natural Killer T-Cells

In one aspect, the invention provides isolated Foxp3+ natural killer T-cells and isolated populations of cells comprising Foxp3+ natural killer T-cells.

Natural Killer T-cells (NKT) cells are thymically derived lymphocytes that express an αβ TCR that recognizes glycolipidic antigens presented by CD1d molecules and receptors from the NK lineage, including NK1.1 and NKG2D (19). Natural Killer T-cells can be classified as Type 1 NKT (invariant), Type 2 NKT and NKT-like. The most studied NKT-cell subset has a semi-invariant TCR comprising an invariant α-chain (Vα14-Jα18 in mice, Vα24-Jα18 in humans) and a restricted TCR-β chain repertoire (Vβ8.2, Vβ7, Vβ2 in mice, Vβ11 in humans). These cells are known as type I, classical or invariant NKT (iNKT) cells, and are able to recognize glycolipids in the context of the MHC class I related molecule CD1d. Although bearing an unique TCR that can be used for their identification, iNKT cells share many surface molecules with T cells, namely CD3 and CD4 in mice, and, in humans, also CD8 (19) (20). Both T-cells and NKT cells develop in the thymus. Positive selection of NKT cells on the basis of their invariant TCR is mediated by double positive thymocytes acting as CD1d⁺ antigen presenting cells, instead of thymic epithelial cells as for conventional T lymphocytes (21). NKT cells are generally considered a lineage separated from T lymphocytes characterized molecularly by the expression of the lineage marker PLZF (22).

iNKT cell subsets which have been identified in vivo present functional properties similar to Th1 cells (producing IFN-γ), Th2 cells (producing IL-4 and IL-13), and, more recently described, Th17 cells (producing IL-17) (1)(2)(3)(4)(5). Interestingly, iNKT cell cytokine production resulting from TCR stimulation does not seem to be influenced by the same transcription factors controlling the functional specialization of conventional T lymphocytes (23).

In one aspect, the invention provides NKT cells that have been converted to express FoxP3. NKT cells that express FoxP3 are referred to herein as Foxp3+ NKT cells, Foxp3+ natural killer T-cells, Foxp3+ Treg NKT cells, NKTreg cells, Natural Killer Treg cells, TregNKT cells, Foxp3+ Treg natural killer T-cells, and Foxp3+ Tregulatory natural killer T-cells. In some embodiments, the invention provides invariant NKT cells (iNKT) cell that have been converted to Foxp3+ cells, also referred to herein as iNKTreg. In some embodiments, the invention provides non-iNKT cells (Type 2 NKT or NKT-like cells) that have been converted to NKT Foxp3+ cells. Foxp3 expression is commonly associated with Treg cells and was previously not found on NKT cells. Treg cells have potent immunosuppressive properties and are thought to prevent pathological self-reactivity (i.e., autoimmune disease) and immune disorders, such as allergy, inflammatory bowel diseases, graft versus host disease and transplant rejection. Interestingly, Foxp3+ NKT cells display a phenotype similar to Foxp3+ Treg lymphocytes: they are CD25⁺, CTLA-4⁺, and GITR⁺ and functionally, they are able to suppress T-cell proliferation with efficiency comparable to Foxp3+ Treg cells.

The Foxp3+ natural killer T-cells described herein have both NKT phenotypes (e.g., markers) and Treg phenotypes (e.g., the FoxP3 marker). In addition, the Foxp3+ natural killer T-cells described herein have a functionality equal to Treg cells (the ability so suppress T-cell proliferation). However, it should be appreciated that Foxp3+ natural killer T-cells can be distinguished from both Foxp3-natural killer T-cells, Treg cells, and indeed any other cell type. Foxp3+ natural killer T-cells can be characterized minimally by identifying at least one marker unique to NKT cells and the FoxP3 marker. In some embodiments, Foxp3+ natural killer T-cells are characterized by their ability to bind CD1d loaded with a glycolipid such as α-GalCer, and have the marker FoxP3. The specific assays for determining the binding of the various NKT cells to CD1d and the various ligands used in these assays are presented further below. In some embodiments, the Foxp3+ natural killer T-cells have any one or any combination of the following marker phenotype: CD25+, CTLA-4+, GITR+, CD103+, IL7-Rα−, CD27−, CD62L−, NK1.1−, DX5−, NKGD2+, and PLZF+. Foxp3+ natural killer T-cells can be either CD4+ or CD4−. It should be appreciated that not all of these the markers have to be present on a cell to be classified as a Foxp3+ NKT cell.

Induction of Foxp3 expression on iNKT cells was coordinated with the up-regulation of a panel of genes also expressed by Foxp3+ Treg cells. Functionally, Foxp3+ NKT cells displayed a strong regulatory potential. However, the adoption of a T cell-specific phenotype was not absolute, with Foxp3+ NKTreg cells retaining some distinctive features. For instance, Foxp3+ iTreg were heterogeneous for the expression of CD62L, indicating that some retain the ability to recirculate to secondary lymphoid organs through the high endothelial venules (HEV). In contrast, NKTreg cells lack CD62L and, thus, would be restricted to intervene in immunologic situations in the periphery

It should be appreciated that the Foxp3+ natural killer T-cells can be characterized by functional assays. Foxp3+ NKT cells have lost the ability to secrete cytokines in the same amount and composition as NKT cells. Foxp3+ natural killer T-cells have the same immunosuppressive properties as Treg cells. Immunosuppressive properties of a cell can be measured, for instance, by the ability of the cell to suppress the proliferation of stimulated CD4+CD25-responder cells. The immunosuppressive function of Foxp3+ NKT cells is thought to be mediated by GITR-GITRL interactions as blocking of this interaction results in the abrogation of the immunosuppressive properties.

The invention embraces isolated Foxp3+ natural killer T-cells, populations that consist only of Foxp3+ natural killer T-cells and populations of cells comprising Foxp3+ natural killer T-cells. The term “isolated” when pertaining to cells or populations of cells, refers to cells or populations of cells that are not in a subject (i.e., human or non-human animal). In some embodiments, the cells are sufficiently separated from other cells or enhanced in cell number versus other cells such that their identity can be confirmed and their properties tested or exploited according to the methods described herein. For instance, isolated cells or cell populations have been harvested from a subject, grown in vitro or have been generated from other cells. In some embodiments, a population of cells comprising Foxp3+ natural killer T-cells has at least 0.001%, at least 0.01%, at least 0.05%, at least 0.1%, at least 0.5%, at least 1%, at least 5%, at least 10%, at least 20%, at least 30%, at least 40%, at least 50%, at least 60%, at least 70%, at least 80%, or at least 90% Foxp3+ natural killer T-cells. In some embodiments, a population of cells comprising Foxp3+ natural killer T-cells has at least 1, at least 10, at least 50, at least 100, at least 500, at least 1,000, at least 5,000, at least 10,000, at least 50,000, at least 100,000, at least 1×10⁶, at least 1×10⁷,or at least 1×10⁸ cells Foxp3+ natural killer T-cells. The remainder of the cells in the population of cells (i.e., the non-FoxP3 cells) may be of any nature. Thus, for instance, the invention embraces populations of Foxp3+ natural killer T-cells and FoxP3⁻ natural killer T-cells, populations of Foxp3+ natural killer T-cells and T-cells (of any subclass). In some embodiments, the population of cells is a combination of Foxp3+ natural killer T-cells and blood cells (of any subclass, such as erythrocytes). In some embodiments, the population of cells is a combination of Foxp3+ natural killer T-cells and non-blood cells. In some embodiments, the population of cells is a combination of Foxp3+ natural killer T-cells, blood cells and non-blood cells. The invention embraces Foxp3+ natural killer T-cells and population of cells comprising Foxp3+ natural killer T-cells of any origin (e.g., animal, such as human or mouse) or derived from any tissue (e.g., spleen, thymus, etc.).

### Generation of Foxp3+ Natural Killer T-Cells

In one aspect, the invention provides methods for generating Foxp3+ natural killer T-cells. In some embodiments, the method for generating Foxp3+ natural killer T-cells comprises contacting a population of cells comprising natural killer T-cells with a combination of TGF-β and one or more NKT-stimulants in amounts sufficient to generate a Foxp3+ natural killer T-cell. In some embodiments, the population of cells is also contacted with IL-2. In some embodiments, the population of cells is also contacted with IL-7, IL-15 and/or IL-21. In some embodiments, the population of cells is also contacted with the neutralizing antibodies anti-IFNγ, anti-IL-4, anti-IL-6, anti-IL 12 and/or anti-IL-27. In some embodiments, the population of cells is a population of blood cells, a population of leukocytes, a population of T-cells, or a population of natural killer T-cells. In some embodiments, the population of cells is harvested from a subject.

Presented herein are methods for the generation of Foxp3+ natural killer T-cells from FoxP3⁻ natural killer T-cells (i.e., “regular” NKT cells). In some embodiments, the generation of Foxp3+ natural killer T-cells is done in vitro. In some embodiments, the generation of Foxp3+ natural killer T-cells is in vivo. In some embodiments, the generation of Foxp3+ natural killer T-cells is done in situ in a specific anatomical location (e.g., organ). The NKT cells from which the Foxp3+ cells are generated can be of any origin. In some embodiments, the NKT cells are murine cells. In some embodiments, the NKT cells are human cells. In some embodiments, the NKT cells are harvested from the liver, from the spleen and from the thymus. In some embodiments, the NKT cells are harvested from the blood.

The methods for the generation of Foxp3+ natural killer T-cells presented herein minimally comprise contacting an NKT cell with one or more NKT stimulants and TGF-β.

The invention embraces the use of any TGF-β or TGF-β analog that result in the expression of FoxP3 in stimulated NKT cells. Thus, TGF-β, as used to generate Foxp3+ natural killer T-cells, includes each of the three isoforms of TGF-β (TGF-β1, TGF-β2, TGF-β3), the protein precursor of TGF-β and the mature TGF-β. TGF-β as used herein also includes variants TGF-β and analogs of TGF-β that have a similar functionality as TGF-β (e.g., cytomodulin-10). The invention also embraces the use of cells that excrete TGF-β. In addition, it should be appreciated that some organs are naturally rich in TGF-β and that, optionally, no additional TGF-β is administered to generate Foxp3+ natural killer T-cells in vivo in these organs. Organs that are naturally TGF-β rich include the gut and the lungs, bone marrow, the liver and organs with certain cancer cells. It should be appreciated that not all cells in an organ need to be rich in TGF-β to provide a TGF-β rich environment that would allow for the conversion of NKT cells to Foxp3+ positive cells. However, generation of Foxp3+ natural killer T-cells in TGF-β rich organs still requires the administration of one or more NKT-stimulants. In some embodiments, the conditions for the generation of Foxp3+ NKT cells from NKT cells includes exposing the cells to at least 1 ng/ml of TGF-β, at least 2 ng/ml of TGF-β, at least 3 ng/ml of TGF-β, at least 4 ng/ml of TGF-β, at least 5 ng/ml of TGF-β, at least 10 ng/ml of TGF-β, at least 100 ng/ml of TGF-β, at least 1 microg/ml of TGF-β, or at least 10 microg/ml of TGF-β. In some embodiments, the generation of Foxp3+ NKT cells is done in vitro and the concentration of TGF-β is at least 10 ng/ml. The concentration of TGF-β will minimally depend on the nature of the composition of the population of cells comprising the NKT cells and the nature of the NKT cells. In all embodiments, a concentration of TGF-β resulting in the expression of FoxP3 in NKT cells is preferred. A person of ordinary skill in the art can readily determine if FoxP3 is expressed and adjust the concentration of TGF-β, if so required.

NKT-cell stimulants are known in the art and include any glycolipid that can be presented by CD (such as α-GalCe) and any agent that can act on a pathway downstream from the receptor, such as an anti-CD3 antibody. NKT stimulants include anti-CD3 antibodies, phytohemaglutinin (PHA), concanavalin A (ConA), phorbol 12-myristate 13-acetate (PMA), ionomycin and TCR agonists such as a CD1d tetramer loaded with a ligand (CD1d ligands include α-GalCer, PBS57, GSL-1, OCH, and others). All glycolipids that can be presented by CD1d can function as NKT stimulants. Furthermore, there are a number of analogues to glycolipids that can stimulate NKT cells. NKT stimulants can also be identified in a functional assay. For instance, NKT cells can be cultured with antigen presenting cells and the putative stimulant and the NKT cells can be assayed for stimulation by evaluating if the NKT cells excrete cytokines such as IL-2, IL-4 and IFN-γ. Stimulation of NKT cells for the in vitro generation of Foxp3+ NKT cells can be performed, for instance, by contacting the NKT cells with an immobilized anti-CD3 antibody, or through the addition of an NKT stimulant to the growth medium of the NKT cells. In addition, Antigen Presenting Cells loaded with any of these NKT stimulants can also be mixed in with the NKT population. Stimulation of NKT cells in vivo can be performed by administration of an NKT stimulant, such as α-GalCer, to a subject. It should be appreciated that the NKT cells can be exposed to multiple NKT stimulants as well. The concentration of NKT stimulant will minimally depend on the nature of the NKT stimulant, the nature of the NKT cells and the composition of the population of cells comprising the NKT cells. In all embodiments, a concentration of NKT stimulant resulting in the stimulation of the NKT cell is preferred. A person of ordinary skill in the art can readily determine if an NKT cell is stimulated and adjust the concentration of NKT stimulant, if so required.

In some embodiments, in addition to TGF-β and one or more NKT stimulants, the NKT cell is also contacted with IL-2. IL-2, as used herein, refers to IL-2 and any variant that has a similar biological function as IL-2. Contacting with IL-2 as used herein is not limited to a specific IL-2 species or specific IL-2 variant. Thus, human NKT cells can be contact with non-human IL-2, as long as the non-human IL-2 is cross-reactive. Furthermore, functional variants of IL-2 have been described (e.g., including amino acid mutations, such as R38A or F42K) and these variants, and functional fragments of IL-2 and these IL-2 variants, are also embraced by the invention. In some embodiments, the cells are exposed to a concentration of IL-2 that is at least 5 ng/ml. In some embodiments, in addition to TGF-β, one or more NKT stimulants, and, optionally IL-2, one or more cytokines or cytokine-neutralizing antibodies are added to the NKT cells. In some embodiments, the NKT cells are contacted with IL-7, IL-15 and/or IL-21 or functional variants and analogs thereof. In some embodiments, the NKT cells are contacted with anti-IFNγ, anti-IL-4, anti-IL-6, anti-IL12 and anti-IL-27. It should be appreciated that the concentration and combination of cytokines and cytokine-neutralizing antibodies may be varied depending on the nature of the NKT cell (e.g., murine or human), origin of the NKT cell (e.g., thymus-derived, blood-derived), the composition of the population of cells comprising the NKT cells, or the nature of the cytokine or neutralizing antibody used. For instance, the combination and concentration of cytokines and neutralizing antibodies may be different in a population comprising mostly NKT cells, or a population of cell comprising mostly non-NKT blood cells and only a small amount of NKT cells. A person of ordinary skill in the art can, according to the methods presented herein, adjust the combination and concentration of cytokines and cytokine-neutralizing antibodies to arrive at a desired level of Foxp3+ NKT cells. In some embodiments, the NKT cells are human cells and the human NKT cells are exposed to at least 10 ng/ml TGF-β, at least 5 microg/ml of anti-IL 12, at least 5 microg/ml of anti-IL-4, at least 5 microg/ml of anti-IFN-γ, at least 2 microg/ml of anti-CD28 and 20 U/ml of IL-2.

Foxp3+ NKT cells can be generated from any population of cells that comprises at least one NKT cell. For instance, Foxp3+ NKT can be generated from peripheral blood, which comprises between 0.001% and 1% of NKT cells. In some embodiments, the peripheral blood is harvested from a subject. In some embodiments, peripheral blood is contacted with an NKT stimulant and TGF-β resulting in the generation of FoxP3 NKT cells. Even though only a small percentage of blood cells are NKT cells, the selective stimulation of NKT cells by exposure to the NKT stimulant will result in the increase in the number of NKT cells in the population of blood cells and therefore in the increase in the population of generated Foxp3+ NKT cells. In some embodiments, the population of blood cells is purified or washed prior to subjecting the cells to TGF-β and an NKT stimulant. Each purification step can result in the removal of population of cells that do not have NKT cells and the percentage of NKT cells in the remainder will increase in percentage therefore. For instance, blood may be centrifuged and the cells washed to remove soluble materials. Likewise, blood may be purified to isolate only leukocytes (which comprise NKT cells) and the leukocytes may subsequently be subjected to TGF-β and an NKT stimulant to convert the NKT cells within the population of leukocytes to Foxp3+ NKT cells. Similarly, blood may be purified further to isolate only T-cells and the population of T-cells which comprises the NKT cells. T-cells, may subsequently be contacted with TGF-β and an NKT stimulant. In some embodiments, NKT cells are purified from the blood cell population and a population of (essentially) NKT cells only is subjected to TGF-β and an NKT stimulant. NKT cells, and population of cells comprising NKT cells (such as T-cells) may also be obtained from non-blood sources. In some embodiments, NKT cells are harvested from the spleen or the thymus or any other organ. In some embodiments, a population of cells (e.g., T-cells) are harvested from the organ and subsequently exposed to TGF-β and an NKT stimulant. If desired, the NKT cells can be purified from the population of organ-harvested cells prior to exposure to TGF-β and an NKT stimulant.

It should be appreciated that the steps of purification and generation of Foxp3+ NKT cells can also be reversed. Thus, a population of blood cells can be contacted with TGF-β and an NKT stimulant and the Foxp3+ NKT cells or all NKT cells (Foxp3+ NKT cells and FoxP3-NKT cells) can subsequently be purified from the remaining blood cells.

The invention is not limited to a specific yield for the methods of the generation of Foxp3+ NKT cells. In some embodiments, at least 10%, at least 20%, at least 30%, at least 40%, at least 50%, at least 60%, at least 70%, at least 80%, at least 90% of the NKT cells is converted in Foxp3+ NKT cells. In some embodiments, at least 40% of the NKT cells is converted in Foxp3+ NKT cells. It should be appreciated that the yield of Foxp3+ NKT cells will depend at least on the nature of the NKT cells (e.g., human vs. mouse, organ-derived vs. blood-derived) and the population of cells comprising the NKT cells. It should also be appreciated that the final percentage of Foxp3+ NKT cells will depend on the initial percentage of NKT cells in the population of cells. Thus, a population of blood cells, with only between 0.001% and 1% of NKT cells, may only comprise a few NKT cells and therefore, after the end of the exposure to NKT stimulant and TGF-β, may only comprise a small percentage of Foxp3+ NKT cells. However, the percentage of NKT cells will likely be higher than the original 0.001% to 1% because only the NKT cells are stimulated, and the NKT cells will therefore grow at a faster rate than any other cells present in the population of cells.

In one aspect, the invention provides methods for increasing the number of Foxp3+ natural killer T-cells starting from a population comprising one or more Foxp3+ natural killer T-cells. In some embodiments, the method comprises contacting a population of cells comprising at least one Foxp3+ natural killer T-cell with a combination of TGF-β, one or more NKT-stimulants, one or more proliferation inducing cytokines and one or more neutralizing antibodies in amounts sufficient to increase the number of Foxp3+ natural killer T-cells. It should be appreciated that this protocol can be practiced both on a population of Foxp3+ natural killer T-cells only, or on a population that comprises both Foxp3+ natural killer T-cells and other cells. If the population of cells comprises non-Foxp3+ NKT cells, it is likely that a number of the NKT cells will be converted in Foxp3+ natural killer T-cells, which can subsequently be expanded. Thus, stimulating a composition of cells including Foxp3+ natural killer T-cells and non-Foxp3+ natural killer T-cells is in effect a combination of increasing the number of Foxp3+ natural killer T-cells though proliferation and conversion. In some embodiments, the proliferation inducing cytokines are IL-2, IL-7, IL-15 and/or IL-21. In some embodiments, the neutralizing antibody is anti-IFNγ, anti-IL-4, anti-IL-6, anti-IL-12 and/or anti-IL-27. It should be appreciated that the desired optimal combination and concentration of proliferation inducing cytokines and neutralizing antibodies will depend on the nature of the Foxp3+ natural killer T-cell, the percentage of Foxp3+ natural killer T-cells and the nature of the non-Foxp3+ natural killer T-cells in the population of cells. In some embodiments, the number of Foxp3+ natural killer T-cells is increased by at least 2-fold, by at least 5-fold, by at least 10-fold, by at least 50-fold, by at least 100-fold, by at least 200-fold, by at least 500-fold, by at least 1000-fold, by at least 10,000-fold, by at least 100,000-fold, by at least 10⁶-fold, by at least 10⁷-fold.

It should be appreciated that the invention includes combinations of the methods for increasing the number of NKT cells, increasing the number of Foxp3+ natural killer T-cells and the methods for converting NKT cells into Foxp3+ natural killer T-cells. Thus, for instance, a population of Foxp3+ natural killer T-cells can be generated by expanding a population of NKT cells followed by conversion of NKT cells to Foxp3+ natural killer T-cells and expansion of the Foxp3+ natural killer T-cells.

Immunosuppressive Properties of Foxp3+ NKT cells

It is shown herein that Foxp3+ NKT cells have immunosuppressive properties similar to Treg cells. Treg cells, which are Foxp3+, have been shown effective in preventing transplant rejection, graft versus host disease (GVHD), allergic diseases, autoimmunity, and other inflammatory-based pathology (24). In fact, those reports made the case for clinical trials of Foxp3+ Treg cells that are currently being conducted (25). Given the similar functional characteristics of Foxp3+ NKTreg and Foxp3+ Treg cells it should be expected that they share the same potential for clinical applications. In addition, NKTreg cells offer an advantage over Treg cells because NKTreg have an invariant TCR. A cell comprising an invariant TCR can more readily be isolated and stimulated than a cell, such as a Treg cell, that does not have an invariant TCR

A consequence of the invariant specificity of NKTreg cells is an antigen non-specific immune regulation. Therefore, Foxp3+ NKTreg cells are likely to have a general immunosuppressive action, as opposed to an antigen specific action, and be useful for therapeutic purposes to which Treg cells have been applied. Polyclonal Treg cell populations are likely to have, at least in part, a similar non-specific immunosuppressive effect, possibly due to cross-reactivity with self-antigens (26). It is this non-specific effect that serves the basis of Treg suppression in lymphopenia-driven proliferation and protection from Graft Versus Host Disease, GVHD (27, 28). GVHD is one of the clinical conditions that allows for a Treg-based intervention and Treg clinical trials have been initiated for this condition (29-32). Organ transplantation can also benefit from apparent non-specific Treg based regulation for the prevention of graft rejection (33, 34). It is likely that by providing some degree of non-specific suppression the natural antigen-specific regulatory mechanisms have an opportunity to reset the immune response towards tolerance.

### Methods for Delivering Foxp3+ NKT Cells to the Liver and Lungs

In one aspect, the invention provides methods for delivering Foxp3+ natural killer T-cells to the liver and methods for suppressing an immune response in the liver. In one aspect, the invention provides methods for delivering Foxp3+ natural killer T-cells to the lung and methods for suppressing an immune response in the lung. In one aspect, the invention provides methods for delivering Foxp3+ natural killer T-cells to mucosal tissue and methods for suppressing an immune response in mucosal tissue. In the foregoing embodiments, the method comprises administering systemically Foxp3+ natural killer T-cells to the subject. In some embodiments, the Foxp3+ natural killer T-cells are autologous cells. In some embodiments, the Foxp3+ natural killer T-cells are generated by contacting natural killer T-cells with an NKT-cell stimulant and TGF-β in amounts sufficient to generate Foxp3+ natural killer T-cells. In some embodiments, the Foxp3+ natural killer T-cells are administered in an amount effective to suppress an immune response in the liver. In some embodiments, the NKT-cell stimulants or TGF-β are administered in an amount effective to induce sufficient amounts of Foxp3+ natural killer T-cells able to suppress an immune response in the mucosa (e.g. gut or lung). In some embodiments, suppressing the immune response in the liver is to treat graft versus host disease, unwanted immune responses associated with or caused by islet transplantation, unwanted immune responses associated with or caused by liver transplant or immune-mediated inflammation to the liver. In some embodiments, immune-mediated inflammation to the liver is autoimmune hepatitis, primary biliary cirrhosis or steatohepatitis. In some embodiments, the Foxp3+ natural killer T-cells are administered in conjunction with islet transplantation or liver transplant. In some embodiments, the genome of the Foxp3+ natural killer T-cell comprises a nucleic acid encoding a polypeptide, wherein the delivery of the Foxp3+ natural killer T-cell to the liver results in the expression of the polypeptide in the liver.

It was surprisingly found herein that upon intravenous delivery of Foxp3+ NKTreg cells into a subject, Foxp3 expression was stably maintained by iNKT cells which accumulate in the liver, but not in lymph nodes. Some Foxp3+ NKTreg cells were also found within the mucosal tissue of the lungs, and initially after administration, in the spleen. Thus, in one embodiment, the invention provides methods for delivering Foxp3+ natural killer T-cells to the liver, lung or spleen, comprising systemically administering Foxp3+ natural killer T-cells. However, it should be appreciated that the Foxp3+ natural killer T-cells can also be administered through local administration directly to the liver, lung or spleen. In some embodiments, the method of delivering Foxp3+ natural killer T-cells to the liver comprises contacting a population of cells comprising natural killer T-cells with one or more NKT-cell stimulants and TGF-β in amounts sufficient to generate Foxp3+ natural killer T-cells. The Foxp3+ natural killer T-cells are subsequently administered systemically. In some embodiments, a pharmaceutical composition comprising Foxp3+ natural killer T-cells is administered. In some embodiments, the Foxp3+ natural killer T-cells are autologous cells. Thus, in some embodiments, the methods comprise, harvesting blood from a subject and contacting the population of blood cells with one or more NKT-cell stimulants and TGF-β in amounts sufficient to generate Foxp3+ natural killer T-cells, and subsequently administering the population of blood cells, now comprising Foxp3+ natural killer T-cells to the subject. It should be appreciated that the population of blood cells can be purified or enriched to increase the number of NKT cells prior to contacting with one or more NKT-cell stimulants and TGF-β. In addition, the population of cells can also be purified after the Foxp3+ natural killer T-cells have been generated, resulting in the increase in the percentage of Foxp3+ natural killer T-cells. In some embodiments, the Foxp3+ natural killer T-cells are expanded prior to administration.

The ability of Foxp3+ natural killer T-cells to home to the liver and lungs allows for the practice of therapeutic methods by using the innate properties of Foxp3+ natural killer T-cells (i.e., immunosuppressive ability) in the liver and lungs. Foxp3+ natural killer T-cells can be administered systemically, for instance by intravenous administration, when it is desired to suppress the immune response in the lung and/or the liver. In addition, the ability of Foxp3+ natural killer T-cells to home to the liver and lungs also allows for the use of the homing properties of these cells to deliver to the lungs and the liver recombinant polypeptides and/or other agents produced by or contained in the cells. Thus, NKTreg cells provide a cellular therapy for immune-mediated liver disease: not only liver autoimmunity or liver transplantation, but the use of the liver as an immune-privileged site for the deposition of immunogenic cells or molecules (i.e., islet transplantation or gene therapy). Furthermore, the liver-specific action reduces off-target effects that would be associated with total-body immune suppression. The liver-specific accumulation of iNKTreg cells is also of therapeutic use for the treatment of liver inflammation, such as associated with transplantation, autoimmune diseases, virus-related inflammatory changes, steatohepatitis, and liver poisoning. In some embodiments, the Foxp3+ natural killer T-cells are administered in an amount effective to suppress an immune response in the liver, wherein suppressing the immune response in the liver is to treat graft versus host disease, unwanted immune responses with islet transplantation, unwanted immune responses associated with or caused by liver transplant or immune-mediated inflammation to the liver. In some embodiments, the Foxp3+ natural killer T-cells are administered in conjunction with islet transplantation or liver transplant.

It should be appreciated that in some embodiments, the homing properties of the Foxp3+ natural killer T-cells are altered. Foxp3+ natural killer T-cells to be administered can be equipped with a cellular organ marker (e.g., cell surface protein), such as kidney marker or gut marker, that facilitates the homing of the cell to a specific organ. Organ markers are known in the art and methods for modifying the Foxp3+ natural killer T-cells to include the marker (e.g., by introducing the nucleic acid encoding the marker in the genome of the cell) are known as well. Thus, in one embodiment, the invention provides a Foxp3+ natural killer T-cell comprising a cell surface protein. In some embodiments the Foxp3+ natural killer T-cells comprising a cell surface protein is generated by introducing into the genome of the Foxp3+ natural killer T-cells a nucleic acid encoding the cell surface protein.

### Delivery of Agents and Polypeptides to the Liver and the Lung

The delivery of Foxp3+ natural killer T-cells with immunosuppressive properties allows for the creation of an immune-privileged site (the liver or lung) without systemic immune suppression. This finding may be therapeutically exploited in combination with other immunogenic therapeutics for the safe delivery of such immunogenic therapeutics into the liver, for instance through the portal vein or by direct administration. Examples are islet transplantation for the treatment of diabetes (routinely administered through the portal vein) and other cell replacement therapies where the cells produce soluble products (such as clotting factors for coagulation disorders, or enzyme replacement therapy for the treatment of lysosomal storage diseases).

In one aspect, the invention provides methods for delivering an agent, (e.g., a therapeutic, a polypeptide, a diagnostic) to the liver or the lungs. In one embodiment, the method comprises modifying a Foxp3+ natural killer T-cell, such that it can deliver an agent to the liver or the lungs. Modification can be done, for instance, by attaching the agent to a cell surface protein or cells surface sugar of the Foxp3+ natural killer T-cell. In addition, the genome of the Foxp3+ natural killer T-cell can be modified to include a nucleic acid encoding a polypeptide that will be expressed when the Foxp3+ natural killer T-cell has migrated to the liver or the lung. Thus, in one embodiment, the invention provided methods for delivering a polypeptide to the liver or lung comprising modifying the genome of the Foxp3+ natural killer T-cell to include a nucleic acid encoding a polypeptide, and administering the Foxp3+ natural killer T-cell comprising the modified genome, wherein administration of the Foxp3+ natural killer T-cell result in the delivery of the Foxp3+ natural killer T-cell to the liver or the lung further resulting in the expression of the polypeptide in the liver or the lung.

In some embodiments, the polypeptide delivered to the liver is a metabolic enzyme. Thus, in one embodiment, the invention provides methods for treating a lysosomal storage disease by delivering a functional metabolic enzyme to the liver, thereby compensating for the metabolic enzyme that is deficient in the subject having the lysosomal storage disease. In one embodiment, the polypeptide delivered to the liver is a polypeptide that has a function in blood homeostasis (e.g., a clotting factor). Thus, in one embodiment, the invention provides methods for treating a blood disorder by delivering a functional polypeptide to the liver that has s function in blood homeostasis.

In some embodiments, the polypeptide delivered to the lung is an enzyme. Thus, in one embodiment, the invention provides methods for lung disorders that can be treated by the administration of a therapeutic polypeptide. For instance, the enzyme dornase alpha can be delivered for the treatment of cystic fibrosis.

In some embodiments, the delivery of an agent, such as a polypeptide, to the liver or the lung is done with autologous cells. Autologous cells will be recognized by the body as “self”, thereby preventing any unwanted immune effects. In one embodiment, blood cells are harvested from a subject and, optionally, purified to increase the number of NKT cells. The NKT cells are subsequently modified to attach an agent to the cell or to include a nucleic acid encoding the desired polypeptide into the genome of the cell. After the NKT cell has been modified, the cell is contacted by TGF-β and an NKT stimulant to convert the cell into a Foxp3+ natural killer T-cell. The modified Foxp3+ natural killer T-cell comprising the attached agent or the nucleic acid encoding the polypeptide can subsequently administered to the subject resulting in the delivery of the modified cell to the liver or lungs. It should be appreciated that the order of the steps can also be changed. For instance, the NKT cells can first be converted into Foxp3+ natural killer T-cells and subsequently be modified to include the nucleic acids or the agent.

### Organ Specific Immune Response

In one aspect, the invention provides methods for in situ generation of Foxp3+ natural killer T-cells in a specific anatomical location, such as an organ, and methods for suppressing an immune response in these locations. In one aspect, the invention provides a method for suppressing an immune response in an organ in a subject, the method comprising delivering locally to the organ one or more NKT-cell stimulants in an amount sufficient to suppress the immune response in the organ. In some embodiments, the method further comprises delivering locally to the organ TGF-β in an amount sufficient to suppress the immune response in the organ. In some embodiments, the immune response is an immune response to an antigen. In some embodiments, the immune response is an autoimmune response. In some embodiments, the organ is the gut. In some embodiments, the suppression of the immune response is to treat inflammatory bowel disease. In some embodiments, the delivering locally to the organ is delivery to mucosal tissue. In some embodiments, the delivering locally to the organ is delivery to mucosal tissue of the lung. In some embodiments, the Foxp3+ NKT cells are generated in the body, following the administration of one or more NKT-cell stimulants if the body site where they are generated contains sufficient amounts of TGF-β to drive their generation.

In some embodiments, the Foxp3+ NKT cells are generated in the body, following the administration of TGF-β if the body site where they are generated contains sufficient amounts of NKT-cell stimulant to drive their generation.

In one aspect, the invention provides a method of in situ generation of Foxp3+ natural killer T-cells in an organ in a subject by delivering locally to the organ one or more NKT-cell stimulants in an amount sufficient to suppress the immune response in the organ. Methods for local delivery of a moiety to a specific organ are known in the art, and are described in more detail below. For instance, it is shown herein that it is possible to induce NKTreg cells in the gut following intra-gastric delivery of the NKT cell agonist α-Galactosylceramide (α-GalCer) in an environment rich in TGF-β. Other examples of organ that are rich in TGF-β are the lungs, liver, bone marrow and certain cancer cells. It should be appreciated that not all cells in these organs may be rich in TNF-β. Not all organs are rich in TGF-β and in situ generation of Foxp3+ natural killer T-cells in organs that are not rich in TGF-β can be done by administering both TGF-β and one or more NKT-cell stimulants locally to the organ. The combination of TGF-β and an NKT stimulant can of course also be delivered to organs that are TGF-β rich. In organs where naturally a sufficient concentration of NKT stimulant is available, only TGF-β would need to be administered to allow for the in situ generation of Foxp3+ natural killer T-cells. The combination of TGF-β and an NKT stimulant can of course also be delivered to organs that are NKT stimulant rich.

The in situ generation of Foxp3+ natural killer T-cells from NKT cells in a specific anatomical location, such as an organ, allows for the induction of an immunosuppressant effect in that organ, without suppressing the immune response in other organs. Thus, for instance, the methods described herein can be used to treat immune related disorders in the gut (e.g., Crohn's disease, inflammatory bowel disease, ulcerative colitis), immune related disorders in the liver, (e.g. autoimmune hepatitis, primary biliary cirrhosis, non-alcoholic and alcoholic steato-hepatitis (NASH and ASH), liver cirrhosis, hepatitis C virus and hepatitis B virus (HCV and HBV)), immune related disorders in the lung (e.g., asthma), inflammation of the central nervous system and arthritis. Local generation of immunosuppressant Foxp3+ natural killer T-cells also allows for the ability to suppress the immune response in any organ or region which is to undergo transplant surgery.

### Detection of Foxp3+ Natural Killer T-Cells

Foxp3+ natural killer T-cells are characterized by their ability to bind glycolipids presented by CD molecules and have the marker Foxp3+. Type II NKT cells can be identified by binding of the cell to CD1d tetramers loaded with sulphatide (See e.g.,. J Exp Med. 2004 Apr. 5;199(7):947-57). Type I NKT cells, also called invariant NKT cells can be identified by binding to CD1d loaded tetramers with one of the following compounds α-galactosyl-ceramide (α-GalCer), PBS-57, OCH, GSL-1, isoglobotrihexosylceramide (iGb3), α-C-galactosylceramide. See also: (http://www.bdbiosciences.com/external_files/pm/doc/tds/dimerx/live/web_enabled/557764.pdf). Type I NKT cells also can be identified by their invariant markers.

CD molecules are non-classical MHC molecules that are characterized as non-polymorphic, conserved among species and possessing narrow, deep, hydrophobic ligand binding pockets. These binding pockets are capable of presenting glycolipids and phospholipids to Natural Killer T (NKT) cells. The best characterized CD1d ligand is α-GalactosylCeramide (α-GalCer), originally derived from marine sponge extract. Presentation of α-GalCer by CD1d molecules results in NKT cell recognition and rapid production of large amounts of IFN-γ and IL-4, bestowing α-GalCer with therapeutic efficacy. More recently, the lysosomal sphingolipid isoglobotrihexosylceramide (iGb3) has been identified as a CD1d ligand. This endogenous sphingolipid is thought to be responsible for NKT cell development. ProImmune provides fluorescently labeled mouse CD1d tetramers pre-loaded with α-GalCer for convenience, or empty for loading with the ligand of choice by the user. Tetrameric CD1d-lipid complexes bind to TCRs of NKT cells of a particular specificity (as determined by the lipid ligand used), allowing identification and enumeration of antigen-specific CD1d-restricted NKT cells by flow cytometry. Additional co-staining for intracellular cytokines (e.g. IFN-γ/IL-2) and/or surface markers (e.g. CD69) can yield functional data for the antigen-specific subset.

PBS-57 is an analogue of α-galactosylceramide recently developed by Dr. Paul Savage and colleagues. Three independent laboratories have shown that PBS-57 activity is indistinguishable from α-galactosylceramide. The NIH Tetramer Facility provides PBS-57 ligand complexed to CD monomers or tetramers.

OCH, an α-galactosylceramide analogue with a truncated side chain, stimulates Th2-biased cytokine production in natural killer T cells. This ligand has been shown to delay the onset of experimental autoimmune encephalomyelitis in an animal model. Purified OCH ligand may also be obtained for stimulation of NK T cells in vitro or for in vivo animal studies. OCH is dissolved in a Tween/sucrose/histidine buffer, sterile-filtered, placed in autoclaved vials, and lyophilized. The resulting powder can be reconstituted in water at a final concentration of 0.2 mg/mL.

Recent studies have shown that glycolipids from the Sphingomonadaceae bacterial family are capable of stimulating natural killer T cells through presentation of the ligands on CD1d molecules. GSL-1 is structurally similar to PBS-57 and α-galactosylceramide. Purified GSL-1 ligand may also be obtained for stimulation of NK T cells in vitro or for in vivo animal studies. GSL-1 is dissolved in a Tween/sucrose/histidine buffer, sterile-filtered, placed in autoclaved vials, and lyophilized. The resulting powder can be reconstituted in water at a final concentration of 0.2 mg/mL.

The α-C-galactosylceramide analogue of α-galactosylceramide is a potent stimulator of natural killer T cells and has been shown to protect animals against certain infections and cancers.

### Simulation of NKT Cells

The invention provides methods for converting NKT cells and expanding populations of NKT cells (including Foxp3+ NKT cells). Methods for stimulating NKT cells are known in the art and include contacting the cells with one or more of the following NKT stimulants: Anti-CD3 antibody (plate-bound or on another surface, such as beads; soluble with antigen presenting cells), Phytohemaglutinin (PHA), Concanavalin A (ConA), Phorbol 12-myristate 13-acetate (PMA)+ionomycin, CD1d presenting specific ligands, described above, as well as these ligands added to CD1d bearing cells (or CD1d coated beads). There are a vast number of analogues to the glycolipids (such as alpha-GalCer) that can also stimulate NKT cells. It should be appreciated that combinations of NKT stimulant can also be used to contact the cells.

### Immune Disorders

In one embodiment, the invention provides methods for generating cells with immunosuppressant properties. In one embodiment the invention provides methods for treating immune disorders using these cells. Immune disorders as used herein include any disease or disorder that has an unwanted immune response, including an autoimmune response and immune responses to allergens. Immune disorders, as used herein, also include unwanted immune responses that may arise in the context of or caused by transplantation, including organ transplantation and the introduction of any desired non-self entity, e.g., cells and proteins, such as used in replacement therapy. Immune disorders include but are not limited to systemic lupus erythematosus (SLE), Sjogren's syndrome, rheumatoid arthritis, juvenile onset diabetes mellitus, Wegener's granulomatosis, inflammatory bowel disease, polymyositis, dermatomyositis, multiple endocrine failure, Schmidt's syndrome, autoimmune uveitis, Addison's disease, adrenalitis, Graves' disease, thyroiditis, Hashimoto's thyroiditis, autoimmune thyroid disease, pernicious anemia, gastric atrophy, chronic hepatitis, lupoid hepatitis, atherosclerosis, presenile dementia, demyelinating diseases, multiple sclerosis, subacute cutaneous lupus erythematosus, hypoparathyroidism, Dressler's syndrome, myasthenia gravis, autoimmune thrombocytopenia, idiopathic thrombocytopenic purpura, hemolytic anemia, pemphigus vulgaris, pemphigus, dermatitis herpetiformis, alopecia arcata, pemphigoid, scleroderma, progressive systemic sclerosis, CREST syndrome (calcinosis, Raynaud's phenomenon, esophageal dysmotility, sclerodactyly, and telangiectasia), adult onset diabetes mellitus (Type II diabetes), male and female autoimmune infertility, ankylosing spondolytis, ulcerative colitis, Crohn's disease, mixed connective tissue disease, polyarteritis nedosa, systemic necrotizing vasculitis, juvenile onset rheumatoid arthritis, glomerulonephritis, atopic dermatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, allergic disease, allergic encephalomyelitis, toxic epidermal necrolysis, alopecia, Alport's syndrome, alveolitis, allergic alveolitis, fibrosing alveolitis, interstitial lung disease, erythema nodosum, pyoderma gangrenosum, transfusion reaction, leprosy, malaria, leishmaniasis, trypanosomiasis, Takayasu's arteritis, polymyalgia rheumatica, temporal arteritis, schistosomiasis, giant cell arteritis, ascariasis, aspergillosis, Sampter's syndrome, eczema, lymphomatoid granulomatosis, Behcet's disease, Caplan's syndrome, Kawasaki's disease, dengue, encephalomyelitis, endocarditis, endomyocardial fibrosis, endophthalmitis, erythema elevatum et diutinum, psoriasis, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, glomerulonephritis, graft versus host disease, transplantation rejection, human immunodeficiency virus infection, echovirus infection, cardiomyopathy, Alzheimer's disease, parvovirus infection, rubella virus infection, post vaccination syndromes, congenital rubella infection, Hodgkin's and Non-Hodgkin's lymphoma, renal cell carcinoma, multiple myeloma, Eaton-Lambert syndrome, relapsing polychondritis, malignant melanoma, cryoglobulinemia, Waldenstrom's macroglobulemia, Epstein-Barr virus infection, mumps, Evan's syndrome, and autoimmune gonadal failure

It should be appreciated that immune disorders, as used herein specifically includes asthma. As used herein, “asthma” refers to a disorder of the respiratory system that is episodic and characterized by inflammation with narrowing of the airways and increased reactivity of the airways to inhaled agents. Asthma is frequently, although not exclusively associated with atopic or allergic symptoms. Symptoms of asthma are widely recognized to include dyspnea, cough, and wheezing; while all three symptoms typically coexist, their coexistence is not required to make a diagnosis of asthma.

In one embodiment, the methods of treating asthma further involve administering an anti-asthma medicament selected from the group consisting of glucocorticoids, beta adrenergic agonists, methylxanthines, anticholinergics, cromolyn, nedocromil, antihistamines, and anti-IgE. In various embodiments the anti-asthma medicament is beclomethasone dipropionate (VANCERIL®, Schering), flunisolide (AEROBID®, Forest), fluticasone propionate (FLOVENT®, GlaxoSmithKline), prednisone, methylprednisolone, triamcinolone acetonide (AZMACORT®, Aventis), albuterol sulfate (VENTOLIN®, GlaxoSmithKline; PROVENTIL®, Schering), epinephrine, isoproterenol hydrochloride, metaproterenol sulfate (ALUPENT®, Boehringer Ingelheim), terbutaline (BRETHINE®, LAMISIL®, Novartis), ipratropium bromide (ATROVENT®, Boehringer Ingelheim), theophylline, cromolyn, nedocromil, or anti-IgE (omalizumab; XOLAIR®; Genentech/Novartis).

### Lysosomal Storage Disorders

In one embodiment the invention provides methods for treating lysosomal storage disorders. Lysosomal storage disorders are caused by lysosomal dysfunction usually as a consequence of deficiency of a single enzyme required for the metabolism of lipids, glycoproteins (sugar containing proteins) or so-called mucopolysaccharides. Lysosomal storage disorders are known in the art and include Activator Deficiency/GM2 Gangliosidosis, Alpha-mannosidosis, Aspartylglucosaminuria, Cholesteryl ester storage disease, Chronic Hexosaminidase A Deficiency, Cystinosis, Danon disease, Fabry disease, Farber disease, Fucosidosis, Galactosialidosis, Gaucher Disease, GM1 gangliosidosis, I-Cell disease/Mucolipidosis II, Infantile Free Sialic Acid Storage Disease/ISSD Juvenile Hexosaminidase A Deficiency, Krabbe disease, Metachromatic Leukodystrophy Mucopolysaccharidoses disorders, Pseudo-Hurler polydystrophy/Mucolipidosis IIIA, MPSI Hurler Syndrome, MPSI Scheie Syndrome, MPS I Hurler-Scheie Syndrome, MPS II Hunter syndrome, Sanfilippo syndrome Type A/MPS III A Sanfilippo syndrome Type B/MPS III B, Sanfilippo syndrome Type C/MPS III C, Sanfilippo syndrome Type D/MPS III D, Morquio Type A/MPS IVA, Morquio Type B/MPS IVB, MPS IX Hyaluronidase Deficiency, MPS VI Maroteaux-Lamy, MPS VII Sly Syndrome, Mucolipidosis I/Sialidosis, Mucolipidosis IIIC, Mucolipidosis type IV, Multiple sulfatase deficiency, Niemann-Pick Disease, Neuronal Ceroid Lipofuscinoses, CLN6 disease, Batten-Spielmeyer-Vogt/Juvenile NCL/CLN3 disease, Finnish Variant Late Infantile CLN5, Jansky-Bielschowsky disease/Late infantile CLN2/TPP1 Disease, Kufs/Adult-onset NCL/CLN4 disease, Northern Epilepsy/variant late infantile CLN8, Santavuori-Haltia/Infantile CLN1/PPT disease, Beta-mannosidosis, Pompe disease/Glycogen storage disease type II, Pycnodysostosis, Sandhoff disease/Adult Onset/GM2 Gangliosidosis, Sandhoff disease/GM2 gangliosidosis—Infantile, Sandhoff disease/GM2 gangliosidosis—Juvenile, Schindler disease, Salla disease/Sialic Acid Storage Disease, Tay-Sachs/GM2 gangliosidosis and Wolman disease

### Blood Disorders

In one embodiment the invention provides methods of treating blood disorders. In one embodiment the blood disorder is a genetic disorder, in which the patient does not have a sufficient amount of a polypeptide needed for blood homeostasis, such as clotting. Blood disorders include, but are not limited to hemophilia, von Willebrand Disease, Bernard-Soulier syndrome, Wiskott-Aldrich syndrome and Glanzmann's thrombasthenia.

### Expression of Polypeptides in Foxp3+ NKT Cells

In one aspect the invention provides methods for delivering a polypeptide to the liver or the lungs. In some embodiments, the genome of Foxp3+ NKT cells or the genome of NKT cells that are to be converted to Foxp3+ NKT cells is modified to include to a nucleic acid encoding the polypeptide to be expressed in the liver or the lungs. Methods for modifying a genome to include a nucleic acid that is to be expressed in the liver or the lungs are known in the art.

The nucleic acid encoding the polypeptide to be expressed by the Foxp3+ cell will be operably joined to regulatory sequences. A coding sequence and regulatory sequences are said to be “operably joined” when they are covalently linked in such a way as to place the expression or transcription of the coding sequence under the influence or control of the regulatory sequences. In order that the coding sequences to be translated into a functional protein the coding sequences are operably joined to regulatory sequences. Two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide.

The precise nature of the regulatory sequences needed for gene expression may vary between species or cell types, but shall in general include, as necessary, 5′ non-transcribing and 5′ non-translating sequences involved with initiation of transcription and translation respectively, such as a TATA box, capping sequence, CAAT sequence, and the like. Especially, such 5′ non-transcribing regulatory sequences will include a promoter region which includes a promoter sequence for transcriptional control of the operably joined gene. Promoters may be constitutive or inducible. Regulatory sequences may also include enhancer sequences or upstream activator sequences, as desired.

A wide variety of transcriptional and translational regulatory sequences may be employed, depending upon the nature of the host. The transcriptional and translational regulatory signals may be derived from viral sources, such as adenovirus, bovine papilloma virus, simian virus, or the like, where the regulatory signals are associated with a particular gene sequence which has a high level of expression. Alternatively, promoters from mammalian expression products, such as actin, collagen, myosin, and the like, may be employed. Transcriptional initiation regulatory signals may be selected which allow for repression or activation, so that expression of the gene sequences can be modulated. Of interest are regulatory signals which are temperature-sensitive so that by varying the temperature, expression can be repressed or initiated, or which are subject to chemical (such as metabolite) regulation.

Expression of the nucleic acid in a subject requires the use of eukaryotic regulatory regions. Such regions will, in general, include a promoter region sufficient to direct the initiation of RNA synthesis. Eukaryotic promoters include, for example, the promoter of the mouse metallothionein I gene sequence (Hamer et al. 1982, J. Mol. Appl. Gen. 1, 273-288); the TK promoter of Herpes virus (McKnight, 1982 Cell 31, 355-365); and the SV40 early promoter (Benoist et al. 1981 Nature (London) 290, 304-310).

It should be appreciated that the regulatory elements for expression of the nucleic acid may be regulatory elements that lead to expression of the nucleic acid in the target issues (e.g, liver and the lungs). Thus, in some embodiments, the nucleic acid is operably connected to a promoter that can express the nucleic acid in a liver or lung environment. Such promoters include promoters for hepatocytes and promoters used in pulmonary cells.

In some embodiments, the nucleic acid is inserted in a vector. As used herein, a “vector” may be any of a number of nucleic acids into which a desired sequence may be inserted by restriction and ligation for transport between different genetic environments or for expression in a host cell. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase or alkaline phosphatase), and genes which visibly affect the phenotype of transformed or transfected cells, hosts, colonies or plaques.

In one embodiment, a vector is employed which is capable of integrating the desired gene sequences into the host cell chromosome. Cells which have stably integrated the introduced DNA into their chromosomes can be selected by also introducing one or more markers which allow for selection of host cells which contain the expression vector. The selectable marker gene sequence can either be directly linked to the DNA gene sequences to be expressed or introduced into the same cell by co-transfection. Additional elements may also be needed for optimal synthesis of the nucleic acid mRNA. These elements may include splice signals, as well as transcription promoters, enhancers, and termination signals. cDNA expression vectors incorporating such elements include those described by Okayama (1983, Molec. Cell. Biol. 3, 280).

Preferred eukaryotic plasmids include, for example, BPV, EBV, SV40, 2-micron circle, and the like, or their derivatives. Such plasmids are well known in the art (1982, Botstein et al., Miami Wntr. Symp. 19, 265-274); Broach, 1981, in: The Molecular Biology of the Yeast Saccharomyces: Life Cycle and Inheritance, Cold Spring Harbor Laboratory, Cold Spring Harbor, N.Y., p. 445-470; Broach, 1982, Cell 28:203-204; Bolton et al. 1980, J. Clin. Hematol. Oncol. 10:39-48; Maniatis, 1980, in: Cell Biology: A Comprehensive Treatise, Vol. 3, Gene Sequence Expression, Academic Press, NY, pp. 563-608). Other preferred eukaryotic vectors are viral vectors. For example, and not by way of limitation, the pox virus, herpes virus, adenovirus and various retroviruses may be employed. The viral vectors may include either DNA or RNA viruses to cause expression of the insert DNA or insert RNA.

Once the vector or DNA sequence containing the construct(s) has been prepared for expression, the DNA construct(s) may be introduced into an appropriate host cell, such as the Foxp3+ NKT cell, by any of a variety of suitable means, i.e., transformation, transfection, conjugation, protoplast fusion, electroporation, calcium phosphate-precipitation, direct microinjection, and the like. After the introduction of the vector, recipient cells are grown in a selective medium, which selects for the growth of vector-containing cells. Expression of the cloned gene sequence(s) results in the production of the nucleic acid. This can take place in the transformed cells as such, or following the induction of these cells to differentiate (for example, by administration of bromodeoxyuracil to neuroblastoma cells or the like).

### Methods for Attaching Agents to Cells

In one aspect the invention provides methods for delivering an agent to the liver or the lungs. In one embodiment the agent is attached to a Foxp3+ NKT cells or NKT cells that are to be converted to Foxp3+ NKT cells. Methods for attaching agent are known in the art and the invention is not limited to any particular method. For instance, an agent can covalently be attached to a cell by reacting the agent with a molecule, such as a sugar or protein that is naturally present on the cell surface. An agent can also be attached to a cell by non-covalently binding the agent to a molecule present on the cell surface. For instance, the agent can be linked to an antibody against a surface protein and the antibody-agent can subsequently be bound to a surface protein. An agent can also be linked to a ligand, such as receptor ligand and the ligand-agent combination can subsequently be attached to the cell. In all embodiments it is preferred that the agent binds to the cell so that the agent does not release from the cell prior to the cell localizing to the liver or the lungs.

Agents that can be attached to the cell include toxins or drugs (i.e., to treat a diseases specific to the liver or lungs), therapeutic polypeptides (i.e., polypeptides, such as lysosomal storage disease enzymes) that have a beneficial effect when delivered to the liver or lungs, and diagnostics.

### Subject

In one aspect, the invention provides methods for the treatment of a disorder in a subject. A “subject”, as used herein, is a human or other vertebrate mammal including, but not limited to, mouse, rat, dog, cat, horse, cow, pig, sheep, goat, or non-human primate.

### Therapeutically Effective Amount

In some embodiments, all compounds, agents and cells described herein (e.g., TGF-β, NKT stimulants, Foxp3+ natural killer T-cells, IL-2, cytokines, anti-cytokine antibodies) can be used in therapeutically effective amounts. The term “therapeutically effective amount” or “effective amount”, which can be used interchangeably, refers to the amount necessary or sufficient to realize a desired therapeutic effect, e.g., suppress the immune response in a specific organ. Combined with the teachings provided herein, by choosing among the various active compounds and weighing factors such as potency, relative bioavailability, subject body weight, severity of adverse side-effects and preferred mode of administration, an effective prophylactic or therapeutic treatment regimen can be selected which does not cause substantial toxicity and yet is effective to treat the particular subject.

The effective amount for any particular application can vary depending on such factors as the disease or condition being treated, the particular compounds, agents and cells described herein to be administered, the size of the subject, or the severity of the disease or condition. One of ordinary skill in the art can empirically determine the effective amount of a particular compound, agent and cell described herein and/or one or more other therapeutic agent without necessitating undue experimentation. It is preferred generally that a maximum dose be used, that is, the highest safe dose according to some medical judgment. Multiple doses per day, week or month may be contemplated to achieve appropriate systemic levels of the compounds, agents and cells described herein. Appropriate system levels can be determined by, for example, measurement of the patient's peak or sustained plasma level of the compounds, agents and cells described herein.

A therapeutically effective amount of a compound or agent typically is between 0.001 and 1000 mg/kg. It is expected that the compounds useful in the present invention will be administered in that range. In some embodiments, the range is 0.01 and 100 mg/kg. In other embodiments, the range is between 0.05 and 50 mg/kg. In some embodiments, a therapeutically effective amount is less than 50 mg/kg, such as less than 45 mg/kg, less than 40 mg/kg, less than 35 mg/kg, less than 30 mg/kg, less than 25 mg/kg, less than 20 mg/kg or less than 15 mg/kg. In some embodiments, a therapeutically effective amount is less than 10 mg/kg, such as less than 9 mg/kg, less than 8 mg/kg, less than 7 mg/kg, less than 6 mg/kg, less than 5 mg/kg, less than 4 mg/kg, less than 3 mg/kg or less than 2 mg/kg. In some embodiments, a therapeutically effective amount is less than 1.5 mg/kg, such as less than 1.4 mg/kg, less than 1.3 mg/kg, less than 1.2 mg/kg, less than 1.1 mg/kg, less than 1 mg/kg, less than 0.9 mg/kg, less than 0.8 mg/kg, less than 0.7 mg/kg, less than 0.6 mg/kg, less than 0.5 mg/kg, less than 0.4 mg/kg, less than 0.3 mg/kg, less than 0.2 mg/kg or less than 0.1 mg/kg of TGF-β, NKT-stimulant or other agent or compound described herein.

A therapeutically effective amount of Foxp3+ natural killer T-cells typically is between 10 and 1×10⁸ cells. In some embodiments, the Foxp3+ natural killer T-cells will be administered in the range of 1×10² and 1×10⁷ cells. In some embodiments, the Foxp3+ natural killer T-cells will be administered in the range of 1×10³ and 1×10⁶ cells. In some embodiments, a therapeutically effective amount is less than 1×10⁷ Foxp3+ natural killer T-cells, such as less than 1×10⁶, less than 1×10⁵, less than 1×10⁴ or less than 1×10³ Foxp3+ natural killer T-cells

In some embodiments, the therapeutically effective amount is administered in one dose. In some embodiments, the therapeutically effective amount is administered in multiple doses. Dosage may be adjusted appropriately to achieve desired levels of the compounds, agents and cells described herein, local or systemic, depending upon the mode of administration. In the event that the response in a subject is insufficient at such doses, even higher doses (or effective higher doses by a different, more localized delivery route) may be employed to the extent that subject tolerance permits. Multiple doses per day are contemplated to achieve appropriate systemic levels of compounds.

### Pharmaceutical Compositions and Routes of Administration

The compounds, agents and cells described herein are typically administered to subjects as pharmaceutical compositions, which may routinely contain pharmaceutically acceptable concentrations of salt, buffering agents, preservatives, compatible carriers, adjuvants, and optionally other therapeutic ingredients. The nature of the pharmaceutical carrier and other components of the pharmaceutical composition will depend on the mode of administration.

The pharmaceuticals compositions of the invention may be administered by any means and route known to the skilled artisan in carrying out the treatment methods described herein.

In some embodiments the compounds, agents and cells described herein are administered locally. Local administration methods are known in the art and will depend on the target area or target organ. Local administration routes include the use of standard topical administration methods such as epicutaneous (application onto the skin), by inhalational, rectal (e.g., by enema or suppository), by eye drops (onto the conjunctiva), ear drops, intranasal route, and vaginal.

Local administration to the gastrointestinal tract can be done by enteral routes of administration. Enteral routes of administration include oral, by gastric feeding tube, by duodenal feeding tube, gastrostomy or rectally.

Local administration can also be performed by infusion. Infusion into specific organs or veins that are in direct contact with specific organs, such as the portal vein, will result, at least initially, to the local administration of the infused entities. In addition to infusion to specific veins, local infusion allows for delivery to the bone marrow (intraosseous infusion), the peritoneum and into the urinary bladder (intravesica infusionl).

Local administration as used herein also includes local injection of the compounds, agents and cells described herein. Local injection can be performed into almost any area or organ and examples of the areas were local administration can be performed are intramuscular, intracerebral, intracerebroventricular, intracardiac, subcutaneous, intradermal, intrathecal, intraperitoneal, and intracavernosal.

It should be appreciated that local administration also includes the use of slow release matrices. Thus, compounds, agents and cells described herein can be introduced into a subject by surgery or injection and the slow release of the entity will facilitate local release of the specific entity.

Local administration as used herein also embraces the use of carriers for local delivery. Thus, compounds, agents and cells described hereinto be locally delivered can be coupled to a carrier, that upon administration, homes that the specific area of the body.

For oral administration, the agents and compounds can be formulated readily by combining the compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally, the oral formulations may also be formulated in saline or buffers, e.g., EDTA for neutralizing internal acid conditions, or may be administered without any carriers.

For oral delivery, the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Examples of the more common inert ingredients that are used as enteric coatings are cellulose acetate trimellitate (CAT), hydroxypropylmethyl-cellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films. A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic powder; for liquid forms, a soft gelatin shell may be used. The shell material of cachets could be thick starch or other edible paper. For pills, lozenges, molded tablets or tablet triturates, moist massing techniques can be used.

The agents and compounds described herein can be included in the formulation as fine multi-particulates in the form of granules or pellets. The formulation of the material for capsule administration could also be as a powder, lightly compressed plugs or even as tablets. The pharmaceutical composition could be prepared by compression. One may dilute or increase the volume of the pharmaceutical composition with an inert material. These diluents could include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the pharmaceutical composition into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Binders may be used to hold the therapeutic agent together to form a hard tablet and include materials from natural products such as acacia, tragacanth, starch and gelatin. An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Glidants that might improve the flow properties of the drug during formulation and to aid rearrangement during compression might be added. The glidants may include starch, talc, pyrogenic silica and hydrated silicoaluminate.

For administration by inhalation, the agents and compounds described herein may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas.

Also contemplated herein is pulmonary delivery of the agents and compounds described herein. The agents and compounds described herein may be delivered to the lungs of a mammal for local or systemic delivery. Other reports of inhaled molecules include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13(suppl. 5):143-146 (endothelin-1); Hubbard et al., 1989, Annals of Internal Medicine, Vol. III, pp. 206-212 (a1-antitrypsin); Smith et al., 1989, J. Clin. Invest. 84:1145-1146 (a-1-proteinase); Oswein et al., 1990, “Aerosolization of Proteins”, Proceedings of Symposium on Respiratory Drug Delivery II, Keystone, Colorado, March, (recombinant human growth hormone); Debs et al., 1988, J. Immunol. 140:3482-3488 (interferon-g and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Nasal delivery of a pharmaceutical composition comprising the agents and compounds described herein is also contemplated. Nasal delivery allows the passage of a pharmaceutical composition of the present invention to the blood stream directly after administering the therapeutic product to the nose, without the necessity for deposition of the product in the lung.

The agents and compounds described herein may also be formulated in rectal or vaginal compositions such as suppositories or retention enemas, e.g., containing conventional suppository bases such as cocoa butter or other glycerides. In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble analogs, for example, as a sparingly soluble salt.

Methods for the administration of cells, including optimized pharmaceutical compositions are known in the art. Cells can be administered by infusion, by injection, such as into the joint, or by surgical insertion. However, the invention is not limited to these embodiments and any method of administration of cells is contemplated.

The pharmaceutical compositions also may comprise suitable solid or gel phase carriers or excipients. Examples of such carriers or excipients include but are not limited to calcium carbonate, calcium phosphate, various sugars, starches, cellulose analogs, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or one or more auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, 1990, Science 249, 1527-1533, which is incorporated herein by reference.

The agents and compounds described herein may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

The pharmaceutical compositions of the invention contain an effective amount of the agents and compounds and cells described herein and optionally additional therapeutic agents included in a pharmaceutically-acceptable carrier. The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

Both non-biodegradable and biodegradable polymeric materials can be used in the manufacture of particles for delivering the compounds of the invention. Such polymers may be natural or synthetic polymers. The polymer is selected based on the period of time over which release is desired. Bioadhesive polymers of particular interest include bioerodible hydrogels described by Sawhney et. al., 1993, Macromolecules 26, 581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The agents and compounds described herein may be contained in controlled release systems. The term “controlled release” is intended to refer to any agents and compounds described herein-containing formulation in which the manner and profile of agents and compounds described herein release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a compound over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the compound there from. “Delayed release” may or may not involve gradual release of a compound over an extended period of time, and thus may or may not be “sustained release.” Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

### Kits

In one aspect the invention provides kits comprising a pharmaceutical composition comprising the agents and compounds described herein and instructions for administration of the pharmaceutical composition. In some aspects of the invention, the kit can include a pharmaceutical preparation vial, a pharmaceutical preparation diluent vial, and the compounds and agents described herein. The diluent vial contains a diluent such as physiological saline for diluting what could be a concentrated solution or lyophilized powder of the compound of the invention. In some embodiments, the instructions include instructions for mixing a particular amount of the diluent with a particular amount of the concentrated pharmaceutical preparation, whereby a final formulation for injection or infusion is prepared. In some embodiments, the instructions include instructions for use in a syringe or other administration device. In some embodiments, the instructions include instructions for treating a patient with an effective amount of the compounds of the invention. It also will be understood that the containers containing the preparations, whether the container is a bottle, a vial with a septum, an ampoule with a septum, an infusion bag, and the like, can contain indicia such as conventional markings which change color when the preparation has been autoclaved or otherwise sterilized.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teaching that is referenced hereinabove.

### EXAMPLES

### Materials and Methods for Examples

### Mice

C57BL/6, Balb/c, TGFβRIIdn, and FoxP3^{gfp} knockin mice (generously provided by A. Y. Rudensky, University of Washington, Seattle, Wash.) were bred and maintained in specific pathogen-free conditions at the Instituto Gulbenkian de Ciência, in Oeiras, Portugal. Experimental mice were sex-matched and between 6 and 8 weeks of age. All experiments were conducted in accordance with guidelines from the Animal User and Institutional Ethical Committee. For in vivo conversion of iNKT cells, 30 μg of α-GalCer (Alexis, San Diego, Calif.) was delivered by intra-gastric gavage three times every other day.

### Organ Processing

All organs analyzed were processed into single-cell suspensions with the aid of BD cell-strainers and the piston of a syringe. Spleens were further incubated for 5 minutes in ice-cold Tris-ammonium chloride red blood cell lysis solution. Livers were washed 3 times in PBS with heparin before processing, and then washed in RPMI supplemented with 10% fetal bovine serum. Liver cells were fractionated by overlaying a 35% (vol/vol) Percoll (Sigma) solution (11 ml) followed by centrifugation at 1360 g for 25 min at RT with no brake. Supernatant was discarded by aspiration and the pellet incubated for 5 minutes in ice-cold Tris-ammonium chloride red blood cell lysis solution.

### Isolation of Human Peripheral Blood Cells

Heparinized venous blood samples were obtained from healthy volunteers of both sexes after informed consent. The procedures were reviewed and approved by the Ethical Board of the Faculty of Medicine, University of Lisbon, Lisbon, Portugal. Peripheral blood mononuclear cells (PBMCs) were isolated by centrifugation on Histopaque-1077 Hybri-Max density gradient (Sigma) and T cells enriched as described below.

### Flow Cytometry and Cell Sorting

Mouse and human CD1d-PBS57 tetramers coupled to PE were supplied by the NIH Tetramer Facility. Fluorochrome-labeled monoclonal antibodies (clone indicated in parentheses) against mouse CD3 (145-2C11), CD4 (GK1.5 and RM4-5), CD8 (53-6.7), CD25 (PC61.5), CD27 (LG.7F9), CD62L (MEL-14), CD103 (2E7), CTLA-4 (UC10-4B9), DX5 (DX5), Foxp3 (FJK-16s), GITR (DTA-1), NK1.1 (PK136), NKG2D (CX5), TCR-β chain (H57-597), Thy1.1 (HIS51), Thy1.2 (53-2.1) were purchased from eBioscience or BD Biosciences. Fluorochrome-labeled monoclonal antibodies against human CD4 (SK3), CD25 (2A3), CD127 (eBioRDR5), CD161 (DX12), CTLA-4 (14D3), Foxp3 (PCH101), GITR (eBioAITR) and TCR Vβ11 (C21) were purchased from eBioscience, BD Biosciences or Beckman Coulter.

For murine NKT cell enrichment, cells were incubated with unconjugated anti-CD 16/32 (clone 2.4G2) rat antibody (in-house production) to block nonspecific binding to FcR and labeled with PE-conjugated CD1d-PBS57 tetramers without washing. Anti-PE magnetic beads were added and the magnetically-labeled fraction was isolated in an autoMACS cell separator (Miltenyi Biotech). Samples were analyzed on a FACSCanto I (Becton Dickinson) or sorted on FACSAria (Becton Dickinson), with doublet exclusion in all experiments. For human NKT cell enrichment, cells were labeled with biotinylated antibodies against CD14 (61D3), CD19 (HIB19) and CD123 (6H6), bound to anti-biotin magnetic beads and enriched on an autoMACS cell separator (Miltenyi Biotech), the magnetically-labeled fraction being discarded. For intracellular flow cytometry stainings, cells were first labeled for surface markers, washed and fixed with the permeabilization and fixation buffer “Foxp3 Staining Buffer Set” from eBioscience. Before intracellular antibody staining, murine cells were incubated with unconjugated anti-CD16/32 (clone 2.4G2) rat antibody (in-house production) and human cells with normal rat serum (eBioscience). Data was analyzed by FlowJo (Tree Star).

### Cell Culture

Sorted mouse cells were cultured in 24- or 96-well flat bottomed plates previously coated with anti-CD3 (clone 145-2C11, eBioscience) at 3 μg/mL. Culture medium was RPMI-1640 with GlutaMAX, supplemented with 10% fetal bovine serum, 1% Hepes, 1% penicillin/streptomycin, 1% sodium piruvate and 0.1% β-mercaptoethanol (Invitrogen). In some conditions, the following cytokines and antibodies were added to the cultures: TGF-β (5 ng/mL, R&D Systems), recombinant IL-1β (10 ng/mL, eBioscience), IL-2 (5 ng/mL, eBiocience), IL-4 (20 ng/mL, eBioscience), IL-6 (20 ng/mL, R&D Systems), IL-15 (100 ng/mL, eBioscience), IL-7 (5 ng/mL, R&D Systems), and anti-CD28 (2 μg/mL, eBioscience). Before intracellular cytokine detection, cells were stimulated with 4-α-phorbol 12-mystrate 13-acetate (PMA) at 50 ng/mL and ionomycin at 500 ng/mL (Sigma) for 3 hours at 37° C., 5% CO₂ in the presence of Brefeldin A (Sigma).

Human cells were cultured in 24-flat bottomed plates previously coated with anti-CD3 (clone OKT3, BD Biosciences) at 1 μg/mL. Culture medium was RPMI-1640 with GlutaMAX, supplemented with 10% fetal bovine serum, 1% Hepes and 1% penicillin/streptomycin (Invitrogen). In some conditions, the following cytokines and antibodies were added to the cultures: TGF-β (10 ng/mL, R&D Systems), recombinant IL-2 (20 U/mL, Roche), anti-IL12 and anti-IFN-γ (5 μg/mL, eBioscience), anti-IL-4 (5 μg/mL, R&D Systems), and anti-CD28 (2 μg/mL, eBioscience).

### Cytokine Detection

Supernatant of cultures was taken from each well and frozen at −80° C. until cytokine detection. ELISA was performed using IL-10 (Peprotech) and IL-4 (BD-Pharmingen) kits. Detection of IL-9 was performed using the cytokine bead-array Mouse IL-9 Flex Set (BD-Pharmingen). All assays were performed according to the manufacturer's instructions.

In vitro Proliferation and Suppression Assays

To track cell proliferation, sorted murine cells were labelled with 5 μM of carboxyfluorescein succinimidyl ester (CFSE, Invitrogen) before culture. To assess the suppression capacity of regulatory iNKT and CD4 T cells, each subset was co-cultured in triplicates or quadruplicates in 60-well Terasaki plates (Greiner) with mitomycin C (Sigma)-treated splenocytes and freshly sorted CD4⁺CD25⁻ T cells, with the addition of 2.5 μg/mL soluble anti-CD3 (BD Pharmingen). In some experiments, 200 μg/mL of anti-IL10R (1B1.2), 200 μg/mL anti-IL-4 (11B11), and 100 μg/mL anti-GITR (YGITR)²² were added. Cells were cultured at 37° C. for 96 h and 1 μCi [³H]thymidine (Amersham) was added to each well in the last 12 h of culture. Plates were harvested onto fiberglass filters and [³H]thymidine incorporation assessed using the Microbeta Trilux scintillation counter (Perkin Elmer). All cultures were tested in triplicate or quadruplicate.

### Transwell Assays

In transmembrane cultures, CFSE-labelled “responder” CD4⁺CD25⁻ T cells were stimulated with mitomycin C-treated splenocytes and 1 μg/mL soluble anti-CD3 antibody in the bottom wells of a flat-bottomed 96-well culture plate. Regulatory populations were cultured either with “responder” cells in the bottom wells or only with mitomycin C-treated splenocytes in the upper well of 0.2 μm Anopore membrane insert (Nunc). CFSE dilution in the bottom well was assessed after 72 hours by flow cytometry analysis.

### Allergic Airways Disease

Balb/c mice were sensitized at days 0 and 14 by i.p. injection of 20 μg of ovalbumin (OVA, grade V; Sigma, St Louis, USA) or β-lactoglobulin (Sigma), previously run through a DetoxyGel column (Pierce, Rockford, USA) following manufacturer instructions, and suspended in 2.0 mg of endotoxin-free aluminum hydroxide (Alu-gel-S, Serva, Heidelberg, Germany). C57/Bl6 mice were sensitized with half the OVA dose at day 0, 7, and 14. All animals were subsequently intranasally challenged with 50 μg of OVA in pyrogen-free saline at the days indicated in FIG. 14, and sacrificed 24 hours after the last challenge. For quantifying BAL eosinophilia the airways were washed through the trachea by slowly infusing and withdrawing 1 ml of cold PBS 10% BSA (Sigma) three times. The BAL was then centrifuged, the supernatant removed, and the pellet resuspended in PBS. The cells were counted with a hemocytometer. Differential cell counts were performed on cytospin samples stained with Giemsa-Wright (Sigma). At least 200 cells from each sample were counted, using blinded slides, to determine the relative frequency of each cell type. For histology, the lungs were perfused with 4% formalin solution (Sigma), collected and sectioned. Staining was performed using hematoxylin/eosin, and mucus containing cells were revealed using a periodic acid-Schiff (PAS) stain. Photographs were taken using a Leica DM2500 microscope and a Leica DFC420 camera.

### Experimental Autoimmune Encephalomyelitis (EAE)

EAE was induced in C57BL/6 mice by injection of myelin oligodendrocyte glycoprotein (MOG)₃₅₋₅₅ peptide (MEVGWYRSPFSRVVHLYRNGK, SEQ ID NO:1) emulsified in CFA and two intravenous injections of pertussis toxin (day 0, 200 ng; day 2, 400 ng). Some mice were treated with two 4 μg doses of α-GalCer on day 0 (emulsified in the MOG₃₅₋₅₅ CFA mixture) and day 4 (i.p.). Disease severity was monitored daily and EAE was graded as follows: score 1, limp tail; score 2, partial hind-leg paralysis; score 3, complete hind-leg paralysis; score 4, front-leg weakness; score 5, moribund.

### Confocal Microscopy

Foxp3-GFP⁺ cells were sorted in the FACSAria (Becton Dickinson), plated on coverslips pre-coated with poly-L-lysine (Sigma) and incubated for 1 h at 37° C. to adhere. Slides were incubated with PE-labelled CD1d/PBS57 tetramer for 1 h at 4° C. and carefully washed with ice-cold PBS. Cells were fixed in PBS 3% paraformaldehyde (Sigma) for 15 minutes at 4° C. and excess fixative was removed by washing with ice-cold PBS. Slides were mounted in DAPI Fluoromount G (Southern Biotech) mounting medium for fluorescence and examined with a laser scanning confocal microscope (LSM 510 META, Carl Zeiss).

### RNA Extraction, RT and PCR

RNA was extracted from 1,000-50,000 FACS-sorted cells with RNeasy Micro Kit (Qiagen) following the manufacturer's instructions, with the exception of cells being directly sorted into RLT buffer. cDNA synthesis was performed using random primers (Invitrogen) and Superscript III reverse transcriptase (Invitrogen). Transcripts were detected with the following primers (purchased from Bonsai Technologies): PLZF (Zbtb16) fwd: cagtttgcgactgagaatgc, (SEQ ID NO:2) rev: ttcccacacagcagacagaa (SEQ ID NO:3) ; Foxp3 fwd: cccaggaaagacagcaacctt; (SEQ ID NO:4) rev: ttctcacaaccaggccacttg (SEQ ID NO:5); EFA1 fwd: acacgtagattccggcaagt (SEQ ID NO:6), rev: aggagccctttcccatctc (SEQ ID NO:7). PCRs were performed using the Power SYBRGreen PCR Master Mix (Applied Biosystems) and the ABI-PRISM 7000 sequence-detection system (Applied Biosystems). All the PCR products were run in agarose gel and validated for the correct size.

### Statistical Analysis

P values were calculated by non-parametric unpaired t-test with Welch's correction.

### Example 1

### iNKT Cells Express Foxp3 after Culture in the Presence of TGF-β

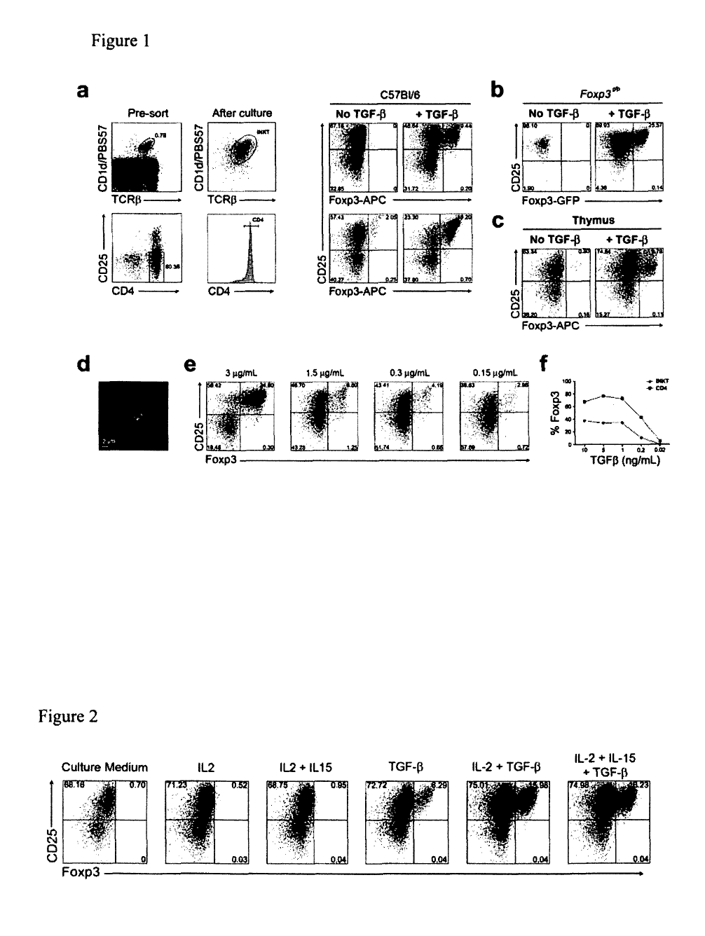

We investigated the plasticity of iNKT in upregulating Foxp3 expression when activated in the presence of TGF-β—a condition known to convert conventional T cells into Foxp3+ “induced” Tregs (35, 36). iNKT cells sorted from the spleen of naïve C57Bl/6 mice were stimulated by plate-bound anti-CD3 and cultured in the presence of IL-2 and TGF-β. Parallel cultures of naïve CD4⁺CD25⁻ T lymphocytes were used as controls. After 3 days, intracellular staining of cultured cells revealed that Foxp3 expression was detectable in a significant proportion of both iNKT (29.35%±11.80) and CD4 (53.21%±12.03) T-cell cultures (FIG. 1a). Similar results were obtained with iNKT cells from mice harboring a GFP-Foxp3 fusion protein-reporter knockin allele (Foxp3^{gfp} mice) (37) and Balb/c mice (FIG. 1b and FIG. 2). iNKT lymphocytes sorted from the thymus could also differentiate into Foxp3+ iNKT cells, yet with lower conversion efficiency (FIG. 1c). Foxp3+ iNKT cells were sorted after conversion and individual cells analyzed by confocal microscopy. As showed in FIG. 1d, the staining with CD1d tetramer loaded with the PBS57 ligand confirms that these Foxp3-expressing cells bear in their surface the invariant TCR that recognizes glycolipid antigens, a feature exclusively attributed to iNKT cells. Therefore, bona fide iNKT cells are similar to conventional CD4 T cells in their ability to upregulate the Foxp3 transcription factor when stimulated under specific conditions. Of note, this property was not shared by other unconventional (non MHC-restricted) T cells, such as γδ T cells, which failed to up-regulate Foxp3 upon activation in the presence of TGF-β (data not shown).

To further define the optimal conditions to convert iNKT cells into Foxp3 expressers, we tested the impact TCR-signal strength, co-stimulation, and cytokine addition. Optimal conversion into Foxp3+ NKT cells was achieved in the presence of 3 μg/mL of plate-bound anti-CD3 and 5 ng/mL of TGF-β and IL-2 (FIG. 1e). Further addition of IL-15 or IL-7 to the previous cytokine cocktail had little impact on iNKT cell conversion (FIG. 2). Conversion in the absence of IL-2 was possible, but the frequency of Foxp3+ cells was greatly decreased (to levels around 5%) possibly due to impaired expansion of those cells. In contrast, no Foxp3+ iNKT cells could be induced in cultures with IL-2 or IL-15 in the absence of TGF-β (FIG. 2). Indeed, titration of TGF-β concentration clearly revealed this cytokine is essential for the induction of Foxp3 expression in iNKT cells (FIG. 1f). We observed some variability in the frequency of converted Foxp3+ iNKT cells (between 20% and 50%) with different TGF-β and fetal bovine serum batches. Addition of anti-CD28 monoclonal antibody to the culture was also tested and did not improve the conversion into Foxp3+ iNKT cells (data not shown). These data clearly demonstrate that TGF-β signals promote the induction of Foxp3 expression upon iNKT-cell activation.

### Example 2

### Foxp3+ iNKT Cells Display Treg and NKT-Cell Phenotypic Characteristics

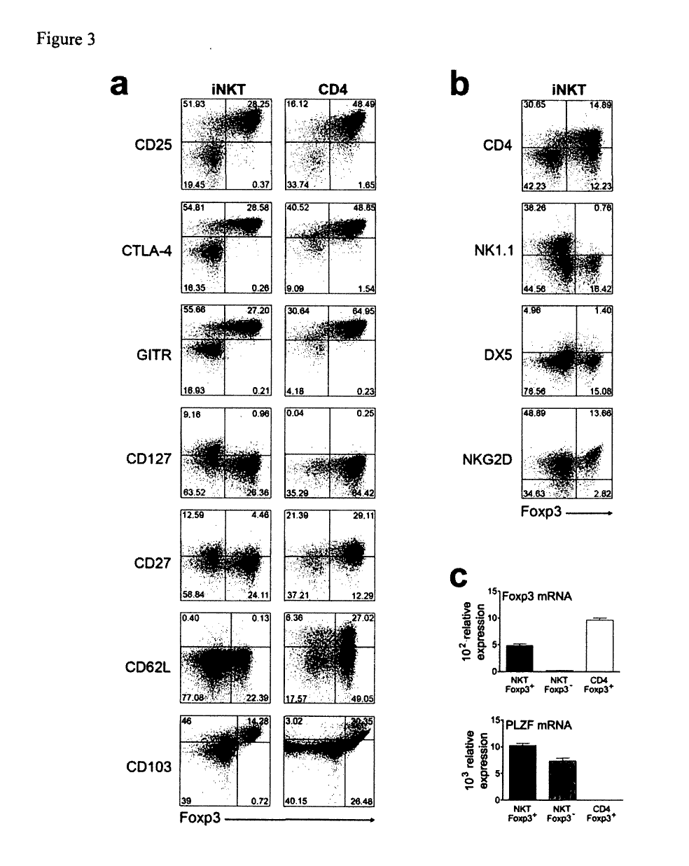

Once we established that iNKT lymphocytes could express Foxp3, we examined the phenotype of the converted cells. Many of the phenotypic characteristics of Foxp3+ iNKT cells were shared with in vitro converted Foxp3+ CD4 Treg cells: both populations were predominantly CD25⁺, CTLA-4⁺, GITR⁺, CD103⁺, and IL-7Rα⁻ (FIG. 3a). However, we observed some differences between the two populations: while Foxp3+ CD4 T cells were predominantly CD27⁺ and heterogeneous for CD62L expression, Foxp3+ iNKT cells were predominantly CD27⁻ and CD62L⁻. The absence of CD62L in association with the high expression of CD103 suggests that, in vivo, Foxp3+ iNKT cells are excluded from the lymph nodes and preferentially migrate to peripheral tissues. Indeed, three weeks after i.v. injection of Foxp3+ iNKT cells into RAG2^{-/-} mice we could detect these cells preferentially in the liver (see below).

Heterogeneity of CD4 expression amongst iNKT lymphocytes is well established⁹. Interestingly, we found the potential to express Foxp3 was not restricted to the CD4⁺ iNKT-cell subset, rather being present in both CD4⁺ and CD4⁻ iNKT cells (FIG. 3b). We also found that the majority of Foxp3+ iNKT lymphocytes were NK1.1⁻, DX5⁻, and NKG2D⁺. In addition, we detected the presence of transcripts for PLZF, a transcription factor reported to be a NKT-lineage signature (22), in sorted Foxp3+ and Foxp3⁻ iNKT cells (FIG. 3c). Together, these observations indicate that induction of Foxp3 expression in iNKT lymphocytes does not corrupt their NKT nature based on the promiscuous expression of molecules from both T and NK lineages.

### Example 3

### Foxp3+ iNKT Cells Migrate to the Liver and Maintain Foxp3 Expression in vivo

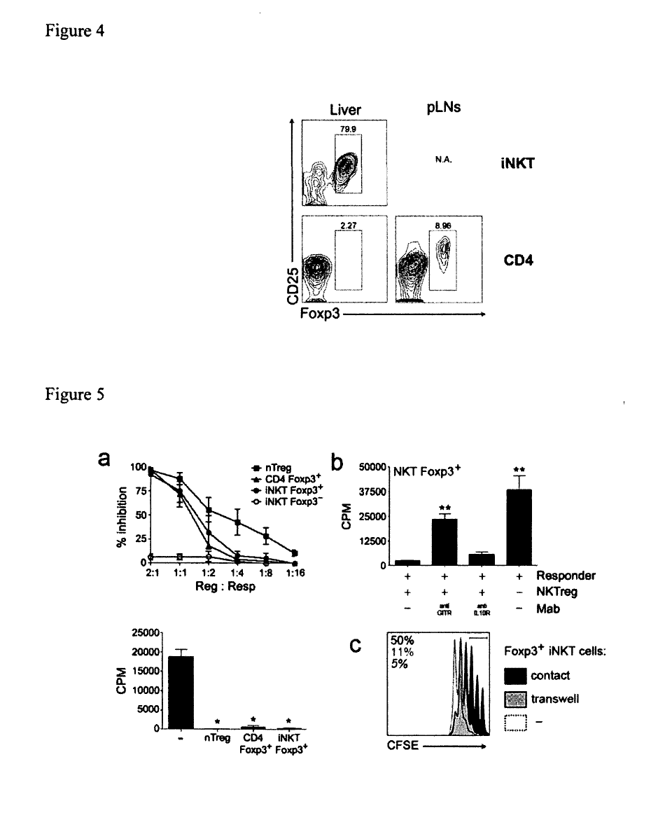

A fundamental aspect to consider after the induction of a gene expression program is its stability. Furthermore, it is becoming apparent that Foxp3 expression by conventional Treg cells is less stable than initially anticipated (38). To investigate if Foxp3 expression could be maintained in iNKT cells in vivo, we injected converted Foxp3^{gfp} iNKT or, as control, in vitro induced Foxp3^{gfp} CD4 cells into RAG2^{-/-} mice. After 7 and 21 days, the phenotype of iNKT cells was assessed in the spleen, lymph nodes (LNs), lungs, gut and liver. In contrast to CD4 T cells, which migrated preferentially to the LNs, but could also be found in the spleen and liver, iNKT cells were absent from LNs, being detected mostly in the liver and lungs, and at early time points, also in the spleen. After three weeks, however, Foxp3+ iNKT cells were no longer detected in the spleen, being instead present in the liver where up to 80% of the iNKT cells still maintained the expression of Foxp3 (FIG. 4). Collectively, these results indicate that in vivo stability of Foxp3 expression by converted iNKT cells is not inferior to converted Foxp3+ Treg cells, although both populations have distinct physiological niches: while iNKT cells preferentially home to non-lymphoid organs, such as liver, CD4 Tregs migrate predominantly to secondary lymphoid organs.

### Example 4

### iNKTreg Cells Display Contact-Dependent GITR-Mediated Suppressive Function

Foxp3 is a transcription factor reported to induce a genetic program in peripheral CD25⁻CD4⁺ T cells, leading to a Treg phenotype and suppressive function (39-41). In order to evaluate the regulatory function of Foxp3+ iNKT cells, we tested their ability to suppress proliferation of CD25⁻CD4⁺ “responder” cells stimulated with APCs and soluble anti-CD3 MAb. We used iNKT cells derived from Foxp3^{gfp} mice converted in the presence of TGF-β and, as controls, natural CD25^{high}CD4⁺ (nTreg) cells and in vitro converted Foxp3+CD4⁺ T cells (also from Foxp3^{gfp} mice). Titration of regulatory to responder-cell ratio revealed that converted Foxp3+ iNKT cells can indeed inhibit the proliferation of target CD25⁻CD4⁺ T cells with similar efficiency to converted Foxp3+CD4⁺ T cells, and only slightly inferior to the efficiency of nTreg cells (FIG. 5a). Addition of anti-GITR (42), but not anti-IL10R, neutralizing antibodies to the cultures reversed the suppression, indicating that GITR plays a predominant role in the regulatory function mediated by Foxp3+ iNKT lymphocytes (FIG. 5b). In agreement with these results, Foxp3+ iNKT cells showed severely impaired suppressive function when cultured separated from responder cells in a transwell (FIG. 5c). These results demonstrate that, similarly to conventional Treg cells, induction of Foxp3 in iNKT lymphocytes endows these cells with suppressive function exerted through a contact-dependent mechanism mediated by GITR.

### Example 5

### In vivo Differentiation of Foxp3+ iNKT Cells is TGF-β-Dependent

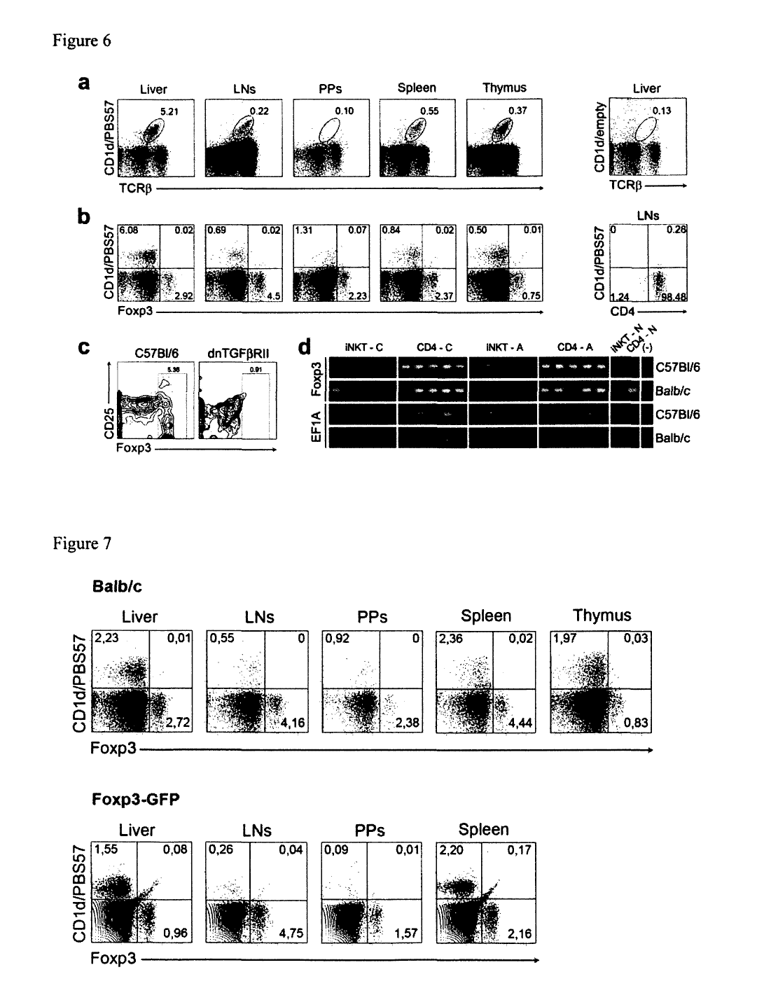

At the time Foxp3^{gfp} mice were generated, the major hematopoietic lineages were screened for the expression of Foxp3. Amongst T and B lymphocytes, NK1.1⁺ cells, macrophages and dendritic cells, Foxp3 expression was observed to be confined to αβ T cells (37). In that study, NKT cells were identified as NK1.1⁺TCRβ⁺ lymphocytes and Foxp3 expression in that cellular subset was ruled out. However, there is a small subset of NKT cells lacking the expression of the NK1.1 receptor. In addition, some subsets of conventional T lymphocytes can express NK 1.1 upon activation. Therefore, we attempted to confirm those observations by identifying unambiguously invariant NKT cells with tetramers that specifically recognize their TCR. We collected mononuclear cells from the liver, spleen, pooled LNs, Peyer's Patches (PP), and thymus of naive C57Bl/6 mice (FIG. 6a,b). While a proportion of CD4⁺ T cells expressed Foxp3, no detectable Foxp3 expression was observed within the iNKT-cell gate from any organ. Similar results were obtained with Balb/c and Foxp3^{gfp} mice (FIG. 7).

We investigated whether gut exposure to α-GalCer could lead to the identification of Foxp3+ iNKT cells in mesenteric lymph nodes (MLNs), in the same way oral tolerance leads to the de novo induction of Foxp3+ Treg cells in MLNs (43). When α-GalCer was delivered by the intra-gastric route we observed an accumulation of Foxp3+ iNKT cells in MLNs, reaching a mean of 1383 (±206, SD) cells per mouse (FIG. 6c). Of note, most of the Foxp3+ iNKT cells from MLNs expressed low levels of CD25. In order to test whether in vivo generation of Foxp3+ iNKT cells requires TGF-β, we observed that Foxp3+ iNKT cells were not induced when α-GalCer was delivered by intra-gastric gavage to dnTGFβRII mice, where T and NKT cells are unable to transduce TGF-β signals (FIG. 6c) (44).

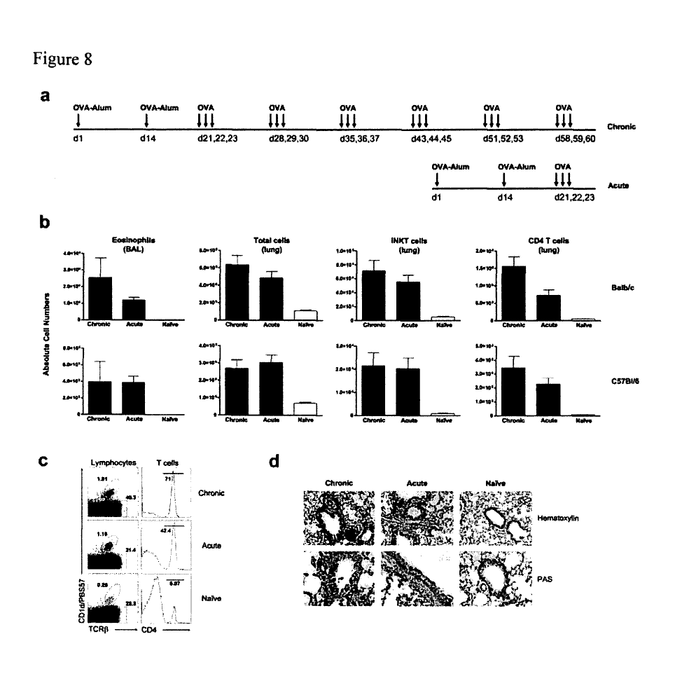

We also explored a mouse model of allergic airways disease where iNKT cells are known to be present in an environment containing cytokines including TGF-β, a key mediator of fibrosis that characterizes tissue remodeling (45). We analyzed Foxp3 expression in iNKT cells, as well as control CD4⁺ T lymphocytes, isolated from the lungs of Balb/c and C57Bl/6 mice sensitized with ovalbumin (OVA) in aluminium hydroxide (alum) i.p. and subsequently challenged with OVA delivered into the airways according to protocols known to lead to acute or chronic allergic airways disease (FIG. 8) (45). We found that Foxp3 was expressed by iNKT cells sorted from the lung of most allergic mice, regardless of acute or chronic disease, although at a lower level than sorted CD4⁺ T cells (FIG. 6d). Of note, unlike CD4 T cells, iNKT cells sorted from lungs of naive, non-manipulated, control mice, did not show Foxp3 expression (FIG. 6d).

Taken together, our observations suggest Foxp3+ iNKT cells are not naturally generated in the thymus, but can be induced in the periphery in environments where TGF-β is present.

### Example 6

### Experimental Allergic Encephalitis (EAE) Protection is Associated with the Local Presence of Foxp3+ iNKTreg Cells

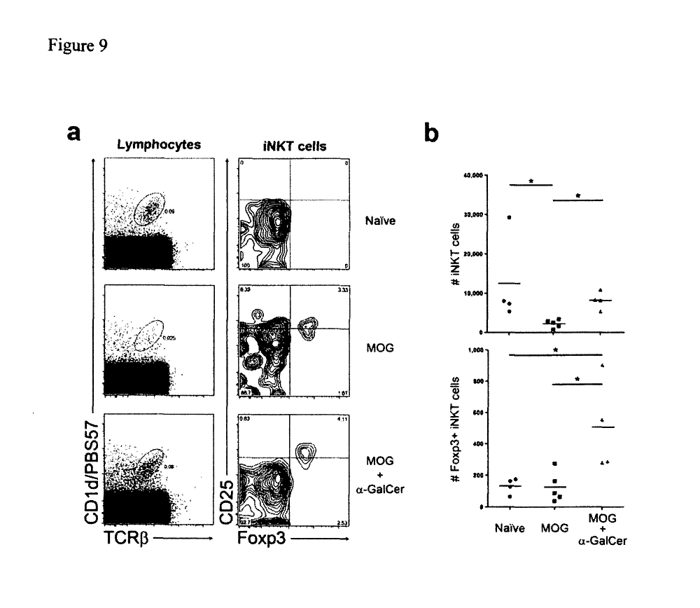

We used an experimental mouse model of multiple sclerosis (MS) to demonstrate whether protective effect previously ascribed to the presence of iNKT cells (46), is associated with the local accumulation of Foxp3+ iNKTreg cells. We found that among the iNKT cells that are recruited to the cervical lymph nodes there is an over-representation of Foxp3+ iNKT cells (FIG. 9). Remarkably, this increase in Foxp3+ iNKT cells is restricted to the central nervous system (CNS)-draining lymph nodes, and not observed systemically (i.e. in the spleen or lymph nodes not draining the CNS). This observation suggests that iNKTreg cells can mediate local anti-inflammatory effects, without leading to overall immune suppression. We also administered α-GalCer to mice subjected to EAE induction. All α-GalCer-treated mice remained protected from the disease, and an increase of Foxp3+ NKTreg cells was observed in the CNS-draining lymph nodes, further confirming our data that it is possible to use NKT cell agonists for the in vivo generation of NKTreg cells, able to prevent immune-mediated diseases.

### Example 7

### Human iNKT Cells can be Converted into Foxp3+ iNKTreg Cells

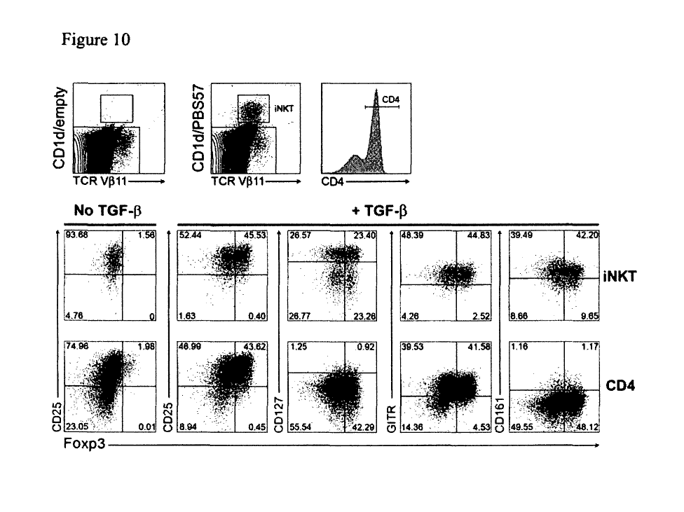

We also evaluated whether Foxp3 expression could be induced in human iNKT cells. Given the lower frequency of iNKT lymphocytes in the human peripheral blood (47), we enriched total T cells by magnetic separation and cultured these bulk populations in polarizing conditions that included not only TGF-β, but also a cocktail of blocking antibodies against IL-12, IFN-γ and IL-4 (conditions described as favoring conversion of human CD4 T cells into Foxp3+ Tregs (48)). After 5 days of culture, up to 40% of human iNKT cells had upregulated Foxp3, an efficiency of conversion comparable to conventional CD4⁺ T cells (FIG. 10). The converted human Foxp3+ NKT cells were CD25⁺, GITR⁺, and predominantly CD161⁺, while CD127 was expressed by approximately half of the Foxp3+ iNKT cells.

### Example 8

### Prevention of Allergic Airway Disease (Asthma) by in situ Foxp3+ iNKT Cell Generation

We use a well established mouse model of allergic airways disease (based on sensitization with ovalbumin (OVA) or the common allergen house dust mite (HDM)) in alum on days 0 and 14, followed by intra-nasal challenge with the allergen on days 20, 21, and 22. We determine whether intra-nasal administration of alpha-GalCer (a NKT cell stimulatory compounds) alone, or in addition to TGF-β, leads to accumulation of NKTreg cells in the airways and prevention of clinical manifestations of the disease. The NKTreg-inducing regimen is administered on the same days as the intra-nasal challenge with the allergen. Reduction of disease severity is assessed by reduced inflammatory infiltrates in histological sections; reduced Th2 cytokines (IL-4, IL-5, IL-13) in lung homogenates; reduced eosinophilic content in the BAL; and importantly reduced airways hyperreactivity, determined by the response (in terms of airways resistance) to increasing doses of inhaled metacholine.

We test different timings of administration in relation with the time of allergen entry in the airways: whether the manifestations can be prevented by prior exposure of the airways to NKTreg inducing treatment; (2) whether disease manifestations can be reduced by administration of the NKTreg inducing treatment at the time of exposure to the allergen and concomitant at a time mice will have overt allergic airways inflammation.

### REFERENCES

1. Kronenberg, M. 2005. Toward an understanding of NKT cell biology: progress and paradoxes. Annual review of immunology 23:877-900.

2. Gumperz, J. E., S. Miyake, T. Yamamura, and M. B. Brenner. 2002. Functionally distinct subsets of CD1d-restricted natural killer T cells revealed by CD1d tetramer staining. The Journal of experimental medicine 195:625-636.

3. Michel, M. L., A. C. Keller, C. Paget, M. Fujio, F. Trottein, P. B. Savage, C. H. Wong, E. Schneider, M. Dy, and M. C. Leite-de-Moraes. 2007. Identification of an IL-17-producing NK1.1(neg) iNKT cell population involved in airway neutrophilia. The Journal of experimental medicine 204:995-1001.

4. Coquet, J. M., S. Chakravarti, K. Kyparissoudis, F. W. McNab, L. A. Pitt, B. S. McKenzie, S. P. Berzins, M. J. Smyth, and D. I. Godfrey. 2008. Diverse cytokine production by NKT cell subsets and identification of an IL-17-producing CD4-NK1.1-NKT cell population. Proceedings of the National Academy of Sciences of the United States of America 105:11287-11292.

5. Rachitskaya, A. V., A. M. Hansen, R. Horai, Z. Li, R. Villasmil, D. Luger, R. B. Nussenblatt, and R. R. Caspi. 2008. Cutting edge: NKT cells constitutively express IL-23 receptor and RORgammat and rapidly produce IL-17 upon receptor ligation in an IL-6-independent fashion. J Immunol 180:5167-5171.

6. Lee, K. A., M. H. Kang, Y. S. Lee, Y. J. Kim, D. H. Kim, H. J. Ko, and C. Y. Kang. 2008. A distinct subset of natural killer T cells produces IL-17, contributing to airway infiltration of neutrophils but not to airway hyperreactivity. Cellular immunology 251:50-55.

7. Niemeyer, M., A. Darmoise, H. J. Mollenkopf, K. Hahnke, R. Hurwitz, G. S. Besra, U. E. Schaible, and S. H. Kaufmann. 2008. Natural killer T-cell characterization through gene expression profiling: an account of versatility bridging T helper type 1 (Th1), Th2 and Th17 immune responses. Immunology 123:45-56.

8. Raftery, M. J., F. Winau, T. Giese, S. H. Kaufmann, U. E. Schaible, and G. Schonrich. 2008. Viral danger signals control CD1d de novo synthesis and NKT cell activation. European journal of immunology 38:668-679.

9. Biburger, M., and G. Tiegs. 2008. Activation-induced NKT cell hyporesponsiveness protects from alpha-galactosylceramide hepatitis and is independent of active transregulatory factors. Journal of leukocyte biology 84:264-279.

10. Moreno, M., J. W. Moiling, S. von Mensdorff-Pouilly, R. H. Verheijen, E. Hooijberg, D. Kramer, A. W. Reurs, A. J. van den Eertwegh, B. M. von Blomberg, R. J. Scheper, and H. J. Bontkes. 2008. IFN-gamma-producing human invariant NKT cells promote tumor-associated antigen-specific cytotoxic T cell responses. J Immunol 181:2446-2454.

11. Akbari, O., P. Stock, E. Meyer, M. Kronenberg, S. Sidobre, T. Nakayama, M. Taniguchi, M. J. Grusby, R. H. DeKruyff, and D. T. Umetsu. 2003. Essential role of NKT cells producing IL-4 and IL-13 in the development of allergen-induced airway hyperreactivity. Nature medicine 9:582-588.

12. Gober, M. D., R. Fishelevich, Y. Zhao, D. Unutmaz, and A. A. Gaspari. 2008. Human natural killer T cells infiltrate into the skin at elicitation sites of allergic contact dermatitis. The Journal of investigative dermatology 128:1460-1469.

13. Mattner, J., P. B. Savage, P. Leung, S. S. Oertelt, V. Wang, O. Trivedi, S. T. Scanlon, K. Pendem, L. Teyton, J. Hart, W. M. Ridgway, L. S. Wicker, M. E. Gershwin, and A. Bendelac. 2008. Liver autoimmunity triggered by microbial activation of natural killer T cells. Cell host & microbe 3:304-315.

14. Jiang, X., T. Shimaoka, S. Kojo, M. Harada, H. Watarai, H. Wakao, N. Ohkohchi, S. Yonehara, M. Taniguchi, and K. Seino. 2005. Cutting edge: critical role of CXCL16/CXCR6 in NKT cell trafficking in allograft tolerance. J Immunol 175:2051-2055.

15. Meyer, E. H., S. Goya, O. Akbari, G. J. Berry, P. B. Savage, M. Kronenberg, T. Nakayama, R. H. DeKruyff, and D. T. Umetsu. 2006. Glycolipid activation of invariant T cell receptor+NK T cells is sufficient to induce airway hyperreactivity independent of conventional CD4+ T cells. Proceedings of the National Academy of Sciences of the United States of America 103:2782-2787.

16. Hong, S., M. T. Wilson, I. Serizawa, L. Wu, N. Singh, O. V. Naidenko, T. Miura, T. Haba, D. C. Scherer, J. Wei, M. Kronenberg, Y. Koezuka, and L. Van Kaer. 2001. The natural killer T-cell ligand alpha-galactosylceramide prevents autoimmune diabetes in non-obese diabetic mice. Nature medicine 7:1052-1056.

17. Singh, A. K., M. T. Wilson, S. Hong, D. Olivares-Villagomez, C. Du, A. K. Stanic, S. Joyce, S. Sriram, Y. Koezuka, and L. Van Kaer. 2001. Natural killer T cell activation protects mice against experimental autoimmune encephalomyelitis. The Journal of experimental medicine 194:1801-1811.

18. Kim, E. Y., J. T. Battaile, A. C. Patel, Y. You, E. Agapov, M. H. Grayson, L. A. Benoit, D. E. Byers, Y. Alevy, J. Tucker, S. Swanson, R. Tidwell, J. W. Tyner, J. D. Morton, M. Castro, D. Polineni, G. A. Patterson, R. A. Schwendener, J. D. Allard, G. Peltz, and M. J. Holtzman. 2008. Persistent activation of an innate immune response translates respiratory viral infection into chronic lung disease. Nature medicine 14:633-640.

19. Bendelac, A., P. B. Savage, and L. Teyton. 2007. The biology of NKT cells. Annual review of immunology 25:297-336.

20. Van Kaer, L. 2007. NKT cells: T lymphocytes with innate effector functions. Current opinion in immunology 19:354-364.

21. Godfrey, D. I., and S. P. Berzins. 2007. Control points in NKT-cell development. Nature reviews 7:505-518.

22. Savage, A. K. et al. 2008. The transcription factor PLZF directs the effector program of the NKT cell lineage. Immunity 29, 391-403.

23. Matsuda, J. L., L. Gapin, J. L. Baron, S. Sidobre, D. B. Stetson, M. Mohrs, R. M. Locksley, and M. Kronenberg. 2003. Mouse V alpha 14i natural killer T cells are resistant to cytokine polarization in vivo. Proceedings of the National Academy of Sciences of the United States of America 100:8395-8400.

24. Allan, S. E., R. Broady, S. Gregori, M. E. Himmel, N. Locke, M. G. Roncarolo, R. Bacchetta, and M. K. Levings. 2008. CD4+ T-regulatory cells: toward therapy for human diseases. Immunological reviews 223:391-421.

25. Brusko, T. M., A. L. Putnam, and J. A. Bluestone. 2008. Human regulatory T cells: role in autoimmune disease and therapeutic opportunities. Immunological reviews 223:371-390.

26. Graca, L., A. Le Moine, S. P. Cobbold, and H. Waldmann. 2003. Dominant transplantation tolerance. Opinion. Current opinion in immunology 15:499-506.

27. Annacker, O., R. Pimenta-Araujo, O. Burlen-Defranoux, T. C. Barbosa, A. Cumano, and A. Bandeira. 2001. CD25+ CD4+ T cells regulate the expansion of peripheral CD4 T cells through the production of IL-10. J Immunol 166:3008-3018.

28. Edinger, M., P. Hoffmann, J. Ermann, K. Drago, C. G. Fathman, S. Strober, and R. S. Negrin. 2003. CD4+CD25+ regulatory T cells preserve graft-versus-tumor activity while inhibiting graft-versus-host disease after bone marrow transplantation. Nature medicine 9:1144-1150.

29. Hoffmann, P., J. Ermann, M. Edinger, C. G. Fathman, and S. Strober. 2002. Donor-type CD4(+)CD25(+) regulatory T cells suppress lethal acute graft-versus-host disease after allogeneic bone marrow transplantation. The Journal of experimental medicine 196:389-399.

30. Cohen, J. L., A. Trenado, D. Vasey, D. Klatzmann, and B. L. Salomon. 2002. CD4(+)CD25(+) immunoregulatory T Cells: new therapeutics for graft-versus-host disease. The Journal of experimental medicine 196:401-406.

31. Taylor, P. A., C. J. Lees, and B. R. Blazar. 2002. The infusion of ex vivo activated and expanded CD4(+)CD25(+) immune regulatory cells inhibits graft-versus-host disease lethality. Blood 99:3493-3499.

32. Hoffmann, P., T. J. Boeld, R. Eder, J. Albrecht, K. Doser, B. Piseshka, A. Dada, C. Niemand, M. Assenmacher, E. Orso, R. Andreesen, E. Holler, and M. Edinger. 2006. Isolation of CD4+CD25+ regulatory T cells for clinical trials. Biol Blood Marrow Transplant 12:267-274.

33. Graca, L., A. Le Moine, C. Y. Lin, P. J. Fairchild, S. P. Cobbold, and H. Waldmann. 2004. Donor-specific transplantation tolerance: the paradoxical behavior of CD4+CD25+ T cells. Proceedings of the National Academy of Sciences of the United States of America 101:10122-10126.

34. Graca, L., A. Le Moine, S. P. Cobbold, and H. Waldmann. 2003. Antibody-induced transplantation tolerance: the role of dominant regulation. Immunologic research 28:181-191.

35. Sakaguchi, S., T. Yamaguchi, T. Nomura, and M. Ono. 2008. Regulatory T cells and immune tolerance. Cell 133:775-787.

36. Chen, W., W. Jin, N. Hardegen, K. J. Lei, L. Li, N. Marinos, G. McGrady, and S. M. Wahl. 2003. Conversion of peripheral CD4+CD25-naive T cells to CD4+CD25+ regulatory T cells by TGF-beta induction of transcription factor Foxp3. The Journal of experimental medicine 198:1875-1886.

37. Fontenot, J. D., J. P. Rasmussen, L. M. Williams, J. L. Dooley, A. G. Farr, and A. Y. Rudensky. 2005. Regulatory T cell lineage specification by the forkhead transcription factor foxp3. Immunity 22:329-341.

38. Komatsu, N. et al. 2009. Heterogeneity of natural Foxp3+ T cells: a committed regulatory T-cell lineage and an uncommitted minor population retaining plasticity. Proceedings of the National Academy of Sciences of the United States of America 106, 1903-1908.

39. Fontenot, J. D., Gavin, M. A. & Rudensky, A. Y. 2003. Foxp3 programs the development and function of CD4+CD25+ regulatory T cells. Nature immunology 4, 330-336.

40. Hori, S., Nomura, T. & Sakaguchi, S. 2003. Control of regulatory T cell development by the transcription factor Foxp3. Science (New York, N.Y 299, 1057-1061.

41. Khattri, R., Cox, T., Yasayko, S. A. & Ramsdell, F. 2003. An essential role for Scurfin in CD4+CD25+ T regulatory cells. Nature immunology 4, 337-342.

42. Tone, M. et al. 2003. Mouse glucocorticoid-induced tumor necrosis factor receptor ligand is costimulatory for T cells. Proceedings of the National Academy of Sciences of the United States of America 100, 15059-15064.

43. Mucida, D. et al. 2005. Oral tolerance in the absence of naturally occurring Tregs. The Journal of clinical investigation 115, 1923-1933.

44. Gorelik, L. & Flavell, R. A. 2000. Abrogation of TGFbeta signaling in T cells leads to spontaneous T cell differentiation and autoimmune disease. Immunity 12, 171-181.

45. Kearley, J., Robinson, D. S. & Lloyd, C. M. 2008. CD4+CD25+ regulatory T cells reverse established allergic airway inflammation and prevent airway remodeling. The Journal of allergy and clinical immunology 122, 617-624 e616.

46. Mars, L. T. et al. 2009. Invariant NKT cells inhibit development of the Th17 lineage. Proceedings of the National Academy of Sciences of the United States of America 106, 6238-6243.

47. Berzins, S. P., Cochrane, A. D., Pellicci, D. G., Smyth, M. J. & Godfrey, D. I. 2005. Limited correlation between human thymus and blood NKT cell content revealed by an ontogeny study of paired tissue samples. European journal of immunology 35, 1399-1407.

48. Mantel, P. Y. et al. 2007. GATA3-driven Th2 responses inhibit TGF-beta1-induced FOXP3 expression and the formation of regulatory T cells. PLoS biology 5, e329.

### EQUIVALENTS

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

The contents of all references, patents and published patent applications cited throughout this application are incorporated herein by reference in their entirety, particularly for the use or subject matter referenced herein.

1. An isolated Foxp3+ natural killer T-cell.

2. An isolated population of cells comprising:
(a) at least 0.001% Foxp3+ natural killer T-cells, or (b) at least 1 Foxp3+ natural killer T-cells.

3. The population of cells of claim 2, wherein a percentage of Foxp3+ natural killer T-cells is at least 0.01%

4. The population of cells of claim 2, wherein the number of Foxp3+ natural killer T-cells is at least 10.

5. The population of cells of claim 2 wherein the population of cells is a population of blood cells, a population of leukocytes, a population of T-cells, or a population of natural killer T-cells.

6. The population of cells of claim 5 wherein the population of cells is a population of T-cells.

7. A method of generating a Foxp3+ natural killer T-cell, the method comprising: contacting a population of cells comprising natural killer T-cells with a combination of
TGF-β and one or more NKT-stimulants in amounts sufficient to generate a Foxp3+ natural killer T-cell.

8. The method of claim 7, further comprising contacting the population of cells with IL-2.

9. The method of claim 7 further comprising contacting the population of cells with any one or any combination of IL-7, IL-15 and IL-21.

10. The method of claim 7 further comprising contacting the population of cells with any one or any combination of neutralizing antibodies selected from the group consisting of anti-IFN[gamma], anti-IL-4, anti-IL-6, anti-IL12 and anti-IL-27.

11. The method of claim 7, wherein the population of cells is a population of blood cells, a population of leukocytes, a population of T-cells, or a population of natural killer T-cells.

12. The method of claim 7 wherein the population of cells is harvested from a subject.

13. A method of increasing the number of Foxp3+ natural killer T-cells, the method comprising: contacting a population of cells comprising at least one Foxp3+ natural killer T-cell with a combination of TGF-β, one or more NKT-stimulants, one or more proliferation inducing cytokines, and one or more neutralizing antibodies in amounts sufficient to increase the number of Foxp3+ natural killer T-cells.

14. The method of claim 13, wherein the number of Foxp3+ natural killer T-cells is increased by at least 2-fold, by at least 5-fold, by at least 10-fold, by at least 50-fold, by at least 100-fold.

15. The method of claim 13, wherein the proliferation inducing cytokine is one or any combination of IL-2, IL-7, IL-15 and IL-21.

16. The method of claim 14, wherein the neutralizing antibody is any one or any combination of anti-IFNγ, anti-IL-4, anti-IL-6, anti-IL12 and anti-IL-27.

17. A method for delivering a natural killer T-cell to the liver or to mucosal tissue in a subject, the method comprising: administering systemically Foxp3+ natural killer T-cells to the subject.

18. A method for delivering a natural killer T-cell to the liver or to mucosal tissue in a subject, the method comprising: administering locally Foxp3+ natural killer T-cells to the subject.

19. The method of claim 17, wherein the Foxp3+ natural killer T-cells are autologous cells.

20. The method of claim 17, wherein the Foxp3+ natural killer T-cells are generated by contacting natural killer T-cells with one or more NKT-cell stimulants and TGF-β in amounts sufficient to generate Foxp3+ natural killer T-cells.

21. The method of claim 17, wherein the Foxp3+ natural killer T-cells are administered in an amount effective to suppress an immune response in the liver or mucosal tissue.

22. The method of claim 21, wherein suppressing the immune response in the liver is to treat graft versus host disease, unwanted immune responses caused by or associated with islet transplantation, unwanted immune responses caused by or associated with liver transplant, or immune-mediated inflammation to the liver.

23. The method of claim 21, wherein the Foxp3+ natural killer T-cells are administered in conjunction with islet transplantation or liver transplant.

24. The method of claim 17, wherein the genome of the Foxp3+ natural killer T-cell comprises a nucleic acid encoding a polypeptide, and wherein the delivery of the Foxp3+ natural killer T-cell to the liver results in the expression of the polypeptide in the liver.

25. A method for suppressing an immune response in an organ in a subject, the method comprising: delivering locally to the organ one or more NKT-stimulants in an amount sufficient to suppress the immune response in the organ.

26. The method of claim 25, further comprising delivering locally to the organ TGF-β in an amount sufficient to suppress the immune response in the organ.

27. The method of claim 25, wherein the immune response is an immune response to an antigen.

28. The method of claim 25, wherein the immune response is an autoimmune response.

29. The method of claim 25, wherein the organ is the gut, or the lungs.

30. The method of claim 29, wherein the suppression of the immune response is to treat inflammatory bowel disease, Crohn's disease, or asthma.

31. The method of claim 25, wherein the delivering locally to the organ is delivery to mucosal tissue.

32. A pharmaceutical composition comprising a population of cells comprising Foxp3+ natural killer T-cells.

33. The pharmaceutical composition of claim 32, wherein the population of cells is a population of blood cells.

34. The pharmaceutical composition of claim 32, wherein the population of cells is a population of leukocytes.

35. The pharmaceutical composition of claim 32, wherein the population of cells is a population of T-cells.

36. The pharmaceutical composition of claim 32, wherein the population of cells is a population of NKT cells.

37. A pharmaceutical composition comprising TGF-β and one or more NKT stimulants.


SEQUENCE LISTING (7 sequences)

SEQ ID NO: 1 - protein, 21 aa, Mus musculus
MEVGWYRSPFSRVVHLYRNGK

SEQ ID NO: 2 - DNA, 20 bp, Mus musculus
cagtttgcgactgagaatgc

SEQ ID NO: 3 - DNA, 20 bp, Mus musculus
ttcccacacagcagacagaa

SEQ ID NO: 4 - DNA, 21 bp, Mus musculus
cccaggaaagacagcaacctt

SEQ ID NO: 5 - DNA, 21 bp, Mus musculus
ttctcacaaccaggccacttg

SEQ ID NO: 6 - DNA, 20 bp, Mus musculus
acacgtagattccggcaagt

SEQ ID NO: 7 - DNA, 19 bp, Mus musculus
aggagccctttcccatctc